(19) United States
(12) Patent Application Publication
(10) Pub. No.: US 2004/0236170 A1
(43) Pub. Date: Nov. 25, 2004

(54) Method for surgically joining a ventricular assist device to the cardiovascular system of a living subject using a piercing introducer assembly

(76) Inventor: Ducksoo Kim, Dover, MA (US)

Correspondence Address: David Prashker, DAVID PRASHKER, P.C., P.O. Box 5387, Magnolia, MA, 01930, US

(21) Appl. No.: 10/786,413

(22) Filed: Feb. 25, 2004

Related U.S. Application Data
(63) Continuation-in-part of application No. 10/748,036, filed on Dec. 29, 2003.
Application No. 10/748,036 is a continuation of application No. 09/713,589, filed on Nov. 15, 2000, now Pat. No. 6,669,708.

(51) Int. Cl. A61M 1/12

(52) U.S. Cl. 600/16; 606/108; 623/3.26

(57) ABSTRACT

The present invention is a unique method for the surgical introduction and implantation in-vivo of a ventricular assist device and the sutureless juncture of prepared inflow and outflow conduits extending from the ventricular assist device to the interior spatial volume of a chosen blood vessel and/or a cardiac chamber within a living subject. The present method employs an introducer assembly which serves as the means for placement of the inflow and outflow conduits of the implanted ventricular assist device.

### PRIORITY FILINGS AND CROSS-REFERENCE

The present application is a Continuation-In-Part of U.S. patent application Ser. No. 10/748,036 filed Dec. 29, 2003, now pending; which is a Continuation of U.S. patent application Ser. No. 09/713,589 filed Nov. 15, 2000, now U.S. Pat. No. 6,669,708 issued Dec. 30, 2003.

### FIELD OF THE INVENTION

The present invention is concerned generally with an improved method for implanting a heart assist device within a living subject, human or animal; and is directed to a surgical method which uses a piercing introducer assembly for the in-vivo introduction and juncture of a ventricular assist device to the cardiovascular system, especially for blood flow between a heart chamber and a chosen blood vessel.

### BACKGROUND OF THE INVENTION

A heart assist device is a mechanical pump device designed for assisting the heart, usually the left ventricle, to pump blood. These devices comprise a pumping chamber and a power source, which may be partially or totally external to the living body and be activated by electromagnetic motors.

A variety of different terms and names are commonly used synonymously and interchangeably, all of them identifying and relating to heart assist devices. Such equivalent and exchangeable terms include:‘ventricular assist device(s)’; ‘artificial ventricle(s)’, ‘ventricular assist pump(s)’, ‘artificial heart ventricle(s)’, ‘heart assist pump(s)’, ‘artificial heart pump(s)’ and ‘vascular assist device(s)’. However, for purposes of clarity and an avoidance of differing nomenclature for the same type of article, the term which is preferred and most often employed herein is “ventricular assist device”. It will be expressly understood and recognized, therefore, that the term “ventricular assist device” refers to, encompasses, and collectively includes all its commonly known synonyms and nomenclature equivalents without reservation or restriction.

One kind of artificial heart pump known as the ABIOCOR TAH is meant to be used as a permanent replacement. The TAH is placed where the heart is located and the heart is surgically excised. This device takes over the functions of the heart.

Ventricular assist devices (VADs and all its synonymous equivalents) are surgically implanted within the thoracic cavity of a living subject to assist the heart to circulate blood. There are two different types conventionally known. A left ventricular assist device (or LVAD) simulates the work of the left ventricle, the main pumping chamber of the heart. A right ventricular assist device (or RVAD simulates the work of the right ventricle. Also, if both patient heart pumping chambers (right and left ventricles) are failing, two heart pumps conventionally known as a biventricular assist device (or BVAD can be used, one pump device being used for each ventricle. The biventricular assist device (BVAD) works similarly to an LVAD, but is used to connect the right ventricle to the pulmonary artery.

The LVAD or BVAD is used with people who have a congestive heart failure (“CHF”) and a weakened left ventricle due to previous heart problems, such as a heart attack, myocarditis, cardiomyopathy, or intra-/post-operative cardiac failure. The left ventricle, which performs about 80% of the heart's work, supplies oxygenated blood to the entire body. If the heart is unable to push blood through the aorta as hard as it should under normal circumstances, this dysfunction/malfunction creates blood circulation and causes the entire body, including the brain, not to receive enough blood. Since blood carries oxygen through the body, this is a serious medical problem, which can cause death if not resolved and eliminated.

Congestive Heart Failure

As a disorder, congestive heart failure (CHF) manifests itself primarily by exertional dyspnea (i.e., difficult or labored breathing) and fatigue. Three medical paradigms are commonly used to describe the causes and therapy of CHF. The first paradigm views this condition in terms of altered pump function and abnormal circulatory dynamics. The other two models describe CHF largely in terms of an altered myocardial cellular performance or as the consequence of an altered gene expression in the cells of the atrophied heart. In its broadest sense, CHF can properly be defined as the inability of the heart to pump blood throughout the body at the rate needed to maintain adequate blood flow, and to maintain many of the normal functions of the body.

It is noteworthy that during the last decade, congestive heart failure (CHF) has burgeoned into the most important public health problem in cardiovascular medicine. As reported by R. F. Gilum [“Epidemiology of Heart Failure in the U.S.”, 126 Am. Heart J. 1042 (1993)], four hundred thousand (400,000) new cases of CHF are diagnosed in the United States annually. The disorder is said to affect nearly 5 million people in this country and about 20 million people worldwide. The number of hospitalizations for CHF has increased more than three fold in the last 15 years.

Unfortunately, nearly 250,000 patients die of heart failure annually. According to the Framingham Heart Study, the 5-year mortality rate for patients with congestive heart failure was 75 per cent in men and 62 per cent in women [Ho, K. K. L., Anderson, K. M., Kannel, W. B., et al., “Survival After the Onset of Congestive Heart Failure in Framingham Heart Study Subject”, 88 Circulation 107 (1993)]. This disorder therefore represents the most common discharge diagnosis for patients over 65 years of age. Also, although the incidence of most cardiovascular disorders has decreased over the past 10 to 20 years, the incidence and prevalence of congestive heart failure has in fact increased at a dramatic rate. This number will continue to increase as those patients who would normally die of an acute myocardial infarction (i.e., heart attack) survive, and as the general population ages.

Non-Surgical Therapeutic Regimens

As a non-surgical regimen of therapeutic treatment, CHF patients are commonly prescribed as many as five to seven different drugs to ameliorate their clinical signs and physical symptoms. These drugs typically include diuretics, angiotensin converting enzyme (ACE) inhibitors, beta-blockers, cardiac glycosides, and peripheral vasodilators. The rationale for pharmacological intervention in heart failure patients include: (i) minimizing the load on the heart; (ii) improving the pumping action of the heart by enhancing the contractility of the muscle fibers; and (iii) suppression of harmful neurohormonal compensatory mechanisms that are activated because of the decreased pumping function of the heart.

Symptoms of congestive heart failure are potentially recurrent and disabling. Almost half of heart failure patients 70 years or older admitted to the hospital will require repeated hospital admission within 90 days. Noncompliance with what is often a complex drug regime may also dramatically adversely affect the recovery of a CHF patient—thus leading to the need for hospitalization, and possibly morbidity and mortality.

Before left ventricular assist devices were available, end-stage heart failure patients had few alternatives when conventional drug therapies failed. It is presently believed that 40,000 persons or more annually could benefit from a human heart transplant, but the shortage of donor organs has imposed a ceiling of about 2,300 transplants a year. This means that many needy people suffering from CHF will die while waiting for a donor heart.

Among the proposed ways to fill this medical gap are artificial hearts and transplants from animals, but each of these alternatives faces significant hurdles: Artificial hearts as a technology are only just entering human field trials after a nearly 20-year break, while researchers have not yet found a way to implant animal hearts into humans without serious risk of organ rejection.

Accordingly, the extreme shortage of donor hearts and the increasing population of patients with ventricular failure suggest that VADs can provide a lifesaving alternative for these patients. The LVAD piggybacks can be surgically implanted onto the patient's own heart; can take over much of the pumping needed for proper blood circulation in-vivo; and can be used in the short term as bridge-to-donor heart transplant. Research investigators have launched a nationwide test to see if VADs can be used as a permanent cure, rather than temporary treatment for CHF, a major step toward an artificial heart resolution of the medical problem.

Efficacy, Safety, and Cost-Effectiveness

A study of the efficacy, safety, and cost-effectiveness of an LVAD for permanent use, compared to optimal medical management through medication therapy was conducted as a clinical trial, namely, the Randomized Evaluation of Mechanical Assistance for the Treatment of Congestive Heart Failure (REMATCH). This trial was conducted in 129 patients at 22 major hospitals throughout the U.S. and was financially supported by the NIH.

The result of this study showed a 48% decrease in the death rate from all causes with the LVAD over the first 2 years of use. Patients in the LVAD group had a median survival of 408 versus 150 days in the medication therapy group. Only 8% (1 out of 12) survived two years in the optimal medical management group. 23% were alive at 2 years in the LVAD group. The quality of life was improved in the LVAD group, based on the questionnaire completed by patients from both groups at one year. The study was conducted on only the sickest patients, who had no alternative options.

As shown by this study, the surgically implanted LVAD virtually eliminated death due to left ventricular heart failure. However, despite the fact that the LVAD could be a better option than medication therapy, the 77% mortality at 2 years is still substantial. The foremost emphasis therefore should still be on preventing the patient's heart from progressing to end-stage heart failure in the first place.

The clinical study indicates that a VAD should be surgically implemented within a short amount of time (weeks or months) after the onset of a myocardial infarction. The considerable disadvantage of this approach, however, is that these implantation techniques can only be performed in a major surgical setting.

Clearly therefore, it would be most advantageous to employ a heart assist system that avoids major invasive surgery and also avoids manipulation of the heart or blood vessels. It would also be beneficial to have such a heart assist system that can be employed in a minimally invasive way and for ease of operating acute heart problems in the patients with severe cardiac dysfunction. Implanting the communicating conduits between cardiovascular system and VAD pump, which currently requires thoracotomy and manipulation of the heart and major vessels such as aorta and pulmonary artery via complex multiple surgical steps. Nevertheless, if such implantation could be done in a less complex way and without major thoracotomy and complex surgical steps, it would provide a simpler and relatively inexpensive means of therapy.

The Current State of the Art

Currently, about 300 to 600 LAVDs are surgically implanted annually. If the temporary heart pump device functions well in these patients—largely the victims of massive heart attacks—the potential target population may approach 32,000 Americans each year, according to some proponents. In contrast, a government official puts the number of people who might benefit from a temporary artificial heart at about 5,000 a year. Thus, depending on whose projected estimate one accepts, the price tag for implanting VADs might range from several hundred million dollars to well over one billion dollars per year.

There remains, however, the long-recognized and continuing need for additional improvements in VAD implantation technique which would allow surgeons to perform more simple VAD procedures in a minimally invasive way. In particular, the need remains for a less invasive method to place one or more communicating or access conduits between the cardiovascular system and the VAD pump without using a heart-lung machine, without stopping the heart, and without using the side biting clamp traditionally employed in these surgical procedures. Were such simplified means to be developed such that the presently existing requirement and necessity of using a complicated surgical method and various instruments are eliminated and avoided, such an improvement would be generally recognized in the medical and surgical arts as a major advance and unusual benefit to both the physician and surgeon as well as his patient.

### SUMMARY OF THE INVENTION

The present invention a method having multiple formats and applications. A preferred format is a method for surgically attaching a ventricular assist device to the circulatory system of a living subject, said method comprising the steps of:

obtaining a vascular assist device comprised of a housing for directing blood flow, a pump able to receive and convey blood, a first conduit of fixed dimensions and configuration having two open ends and at least one internal lumen for directing the flow of blood to said pump, a second conduit of fixed dimensions and configuration having two open ends and at least one internal lumen for conveying blood from said pump, and a source of power for the operation of said pump;

joining a linking connector to an end of said first conduit to form a prepared conduit for accessing and directing blood flow, said linking connector being of determined dimensions and configuration, being deformable on-demand, and being suitable for passage through an aperture;

acquiring a piercing introducer assembly suitable for the introduction of a prepared conduit to the vascular system of a living subject, said introducer assembly comprising:

a perforator instrument comprised of

(i) at least one elongated supporting shaft of predetermined overall dimensions and axial configuration,

(ii) a handle attached at one end to said supporting shaft; and

(iii) a perforating headpiece integrally joined to the other end of said supporting shaft, said perforating headpiece comprising a perforating tip, a penetrating body, and a base aspect, and

(iv) conduit controlling means disposed adjacent to said perforating headpiece on said supporting shaft of said perforator instrument;

attaching said linking connector of said prepared conduit to a chamber of the heart in the living subject using said piercing introducer assembly such that said linking connector of said prepared inflow conduit passes through an aperture in the heart and deforms within a chamber of the heart, thereby securing said conduit to the interior of heart chamber and placing said secured conduit in blood flow communication with the interior of the heart chamber;

surgically affixing said second conduit to the vascular system of the living subject through a surgical means; and

connecting an end of said secured conduit and an end of said surgically affixed conduit to said pump of said ventricular assist device.

### BRIEF DESCRIPTION OF THE FIGURES

The present invention may be better appreciated and more easily understood when taken in conjunction with the accompanying drawing, in which:

FIG. 1 is an illustration of a surgically implanted ventricular assist device within the body of a living subject;

FIG. 2 is a view of an assembled ventricular assist device commercially known as the HeartMate pump:

FIG. 3 is a view of the pump used within the ventricular assist device of FIG. 2;

FIG. 4 is a view of the inflow conduit used within the ventricular assist device of FIG. 2;

FIG. 5 is a view of the outflow conduit used within the ventricular assist device of FIG. 2;

FIG. 6 is a view of the assembled ventricular assist device commercially known as the Micromed DeBakey LVAD:

FIG. 7 is an illustration of the inducer/impeller design used within the ventricular assist device of FIG. 6;

FIG. 8 is a perspective illustration of a first preferred embodiment of the introducer assembly comprising the present invention;

FIGS. 9A and 9B are illustrations of the perforator instrument comprising a component part of the introducer assembly of FIG. 8;

FIGS. 10A and 10B are illustrations of the volumetric sheath comprising a component part of the introducer assembly of FIG. 8;

FIGS. 11A and 11B are illustrations of the position holding means comprising a component part of the introducer assembly of FIG. 8;

FIG. 12 is an illustration of the inter-relationship between the volumetric sheath of FIGS. 10A and 10B and the position holding means of FIGS. 11A and 11B;

FIGS. 13A, 13B, and 13C are illustrations of a linking connector and tubular conduit which comprise a prepared communication or access conduit to be used with the introducer assembly of FIG. 8;

FIG. 14 is an illustration of the inter-relationship between the prepared communication or access conduit of FIGS. 13B and 13C and the perforator instrument of FIGS. 9A and 9B;

FIGS. 15A and 15B are perspective and partial cross-sectional illustrations of the prepared communication or access conduit of FIGS. 13B and 13C when positioned within and part of the complete introducer assembly of FIG. 8;

FIG. 16 is an illustration of the complete introducer assembly of FIGS. 15A and 15B when approaching a sidewall of a blood vessel or cardiac chamber in-vivo;

FIG. 17 is an illustration of the complete introducer assembly after piercing and penetrating through an aperture in the sidewall of a blood vessel or cardiac chamber;

FIG. 18 is an illustration of the advancement forward of the prepared communication or access conduit into the internal spatial volume of a blood vessel or cardiac chamber using the complete introducer assembly;

FIG. 19 is an illustration of the deployment in-situ and the sutureless securing of the prepared communication or access conduit within the internal spatial volume of a blood vessel or cardiac chamber;

FIG. 20 is an illustration of the partial rearward withdrawal of the introducer assembly after the communication or access conduit has been deployed and secured to a blood vessel or cardiac chamber;

FIG. 21 is an illustration of the joined and secured communication or access conduit after the introducer assembly has been removed;

FIG. 22 is an illustration of one alternative embodiment for the perforating headpiece of the perforator instrument of FIG. 9;

FIG. 23 is an illustration of a second alternative embodiment for the perforating headpiece of the perforator instrument of FIG. 9;

FIGS. 24A and 24B are cross-sectional and perspective illustrations of the perforating headpiece of FIG. 23;

FIG. 25 is an illustration of one alternative embodiment for the volumetric sheath of FIG. 10;

FIG. 26 is an illustration of the relationship between the prepared communication or access conduit of FIG. 13C when used in the perforating headpiece of FIGS. 23 and 24 and the volumetric sheath of FIG. 25;

FIG. 27 is an illustration of a second alternative embodiment for the volumetric sheath of FIG. 10;

FIG. 28 is an illustration of one alternative embodiment of the introducer assembly of FIG. 8;

FIG. 29 is a detailed partial cross-sectional illustration of the alternative introducer assembly of FIG. 28;

FIGS. 30A and 30B are illustrations of a first linking connector;

FIGS. 31A and 31B are illustrations of a second linking connector;

FIGS. 32A and 33B are illustrations of a third linking connector;

FIGS. 34A and 34B are illustrations of a fourth linking connector;

FIGS. 35A and 35B are illustrations of an unbranched tubular conduit;

FIG. 36 is an illustration of a multi-branched tubular conduit;

FIGS. 37A and 37B are illustrations of a first type of tubular conduit construction;

FIGS. 38A and 38B are illustrations of a second type of tubular conduit construction;

FIGS. 39A and 39B are illustrations of a third type of tubular conduit construction;

FIGS. 40A and 40B are illustrations of a fourth type of tubular conduit construction;

FIG. 41 is a cross-sectional illustration of a first style of internal lumen for a tubular conduit;

FIG. 42 is a cross-sectional illustration of a second style of internal lumen for a tubular conduit;

FIG. 43 is a cross-sectional illustration of a third style of internal lumen for a tubular conduit; and

FIG. 44 is a cross-sectional illustration of a fourth style of internal lumen for a tubular conduit.

### DETAILED DESCRIPTION OF THE INVENTION

The present invention is an improved surgical method and technique for introducing communication conduits to receive and convey blood between an implanted ventricular assist device (VAD) and a living subject's cardiovascular system, such as between a cardiac chamber and an adjacently positioned blood vessel. The present invention can utilize a synthetic tubular conduit as a communication channel or conduit for receiving and transferring blood in-vivo; or employ a previously excised vascular segment as a tubular conduit to direct blood flow; or use any other biological conduit created via hormonally induced or genetically modified cellular means. In addition, the present invention employs an introducer assembly and system of graft juncture with the prepared communication conduits to create single or multiple conduit communications to receive and convey blood in-vivo. The grafted tubular conduits are used to carry blood between the cardiovascular system and VAD in order to maintain proper blood circulation in the living body.

A number of substantial advantages and major benefits are therefore provided by the present invention, some of which include the following:

1. The present invention provides the means for surgeons to perform single or multiple communication or access conduits or communication or access conduits in-vivo for VAD pump in a minimally invasive manner. The methodology permits the surgeon to utilize either synthetic tubular conduits as communication conduit or previously excised veins or arteries or other biological conduit as communication conduits; and allows the surgeon to place each of the tubular conduits between the LAD pump and cardiovascular system such as cardiac chambers and blood vessels without using a heart-lung machine, without need for stopping the heart, and without major surgical procedures such as thoracotomy or via the mini-thoracotomy during the surgery.

2. The present methodology also avoids the prior need to exclude blood from the section of the blood vessel to which the conduit is being attached. For example, there would be no further need for an aortic side biting clamp—a device with rough semi-circular jaws that isolates a centrally located zone of the aorta from the blood and blood pressure then present in the rest of the aorta.

3. The present invention simplifies the complexity of conventional VAD surgery and makes the surgery less invasive. Moreover, the introducer assembly and technique provides the ability to create multiple communication or access conduit using a minimally invasive procedure which not only shortens the conventional operation time for surgery but also makes the surgery safer and more cost effective.

4. The present invention is suitable for creating a single conduit communication or multiple conduit communication in any medical situation, condition, or pathology in which there is a need for to direct blood flow to a specific blood vessel, vascular area or body region or VAD pump. The cause or source of the medical problem may be congestive heart failure, congenital heart disease, or acquired heart disease involving various cardiovascular systems. Each of these medical conditions has its particular cause, origin, or source; and each of these pathologies, though different in origin, causes a similar effect overall. Accordingly, the present invention is deemed useful and desirable to overcome any of these particular medical conditions and instances where there is a demonstrated need for maintain or increased blood pressure and blood volume flow within a particular part of the cardiovascular system in the body using a VAD pump.

5. The present apparatus and methodology can be employed to create a communication conduit between any cardiovascular system and VAD pump. In many instances, the communication conduit connections will be made between particular parts of the cardiovascular system and a VAD pump, a typical example being conduits between the right or left ventricle and a VAD pump, or between the pulmonary artery or aorta and a VAD pump. However, a communication conduit may also be created between any two veins or between an artery and a vein between the different chambers of the heart, or between the heart chambers and blood vessels in conjunction with any circulatory pump. Equally important, although the primary focus of the present invention is the thoracic cavity and the recognized need for communication conduits among the blood vessels found therein, the present apparatus and methodology may be employed anywhere in the human body where there is a need for increased or maintain blood circulation of the systemic or local region. The sole limitation, therefore, is a means of access for the catheter apparatus, the introducer system, and the methodology to be performed by the skilled surgeon, or interventional cardiologist, or other medical specialist.

Accordingly, in order to better appreciate and more clearly understand the surgical method as well as the means for grafting communication conduits to access, receive and convey blood, the invention as a whole will be disclosed in a format which recites and reveals both the requisite and optional elements and limitations in detail.

### I. A Typical Ventricular Assist Device (VAD)

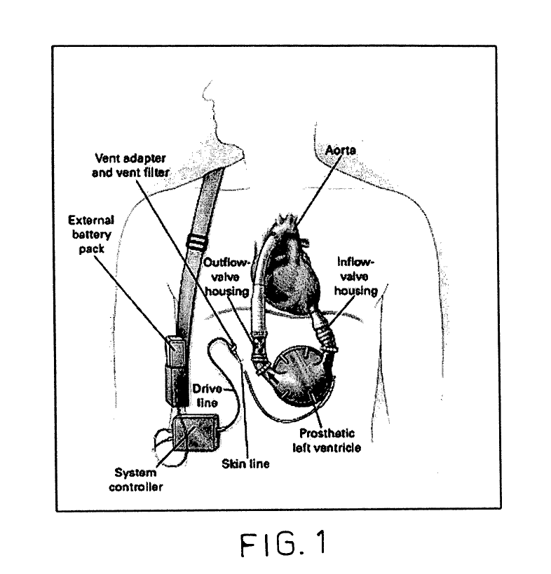

For descriptive purposes only, a typical ventricular assist device (VAD) is described in detail below and is illustrated in-vivo as a surgically implanted article by FIG. 1. It will be clearly understood, however, that the device described and illustrated herein is merely representative of conventional VADs generally; and that the scope of the present invention is not limited in the range of assist devices nor in the intended scope of its applications by the details of the VAD description presented below.

The typical VAD comprises a housing for directing blood flow, an inflow conduit connected to the left ventricle; a small pump implanted underneath the diaphragm; a outflow conduit connected to the aorta (or other blood vessel); and an outside power source (battery). When each of the two conduits (or inflow and outflow communication channels) of the VAD are individually surgically joined and attached to the left ventricle of the patient's heart and the aorta respectively, the result is that device and arrangement illustrated by FIG. 1.

The essential components parts of a VAD are typically shared and held in common by the different manufactures of ventricular assist devices. Accordingly, the individual components of one such VAD (the “HeartMate” pump) are presented below as being merely representative and exemplary of all such devices generally.

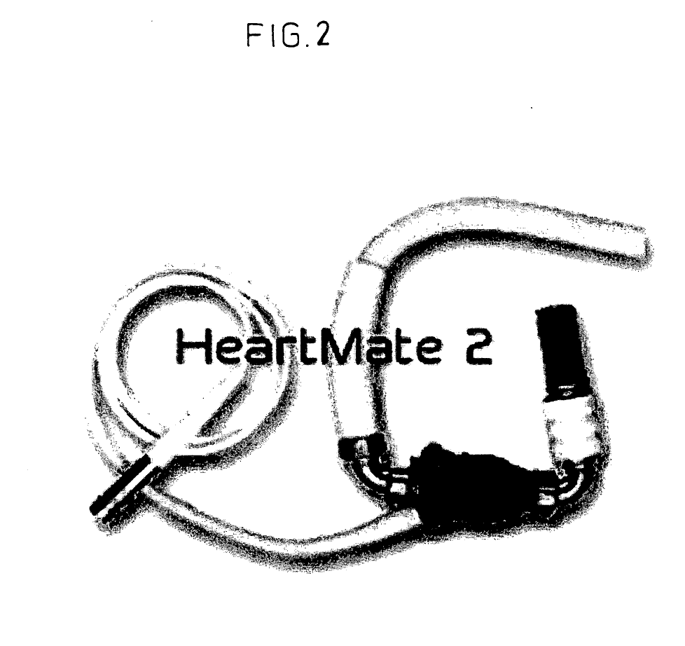

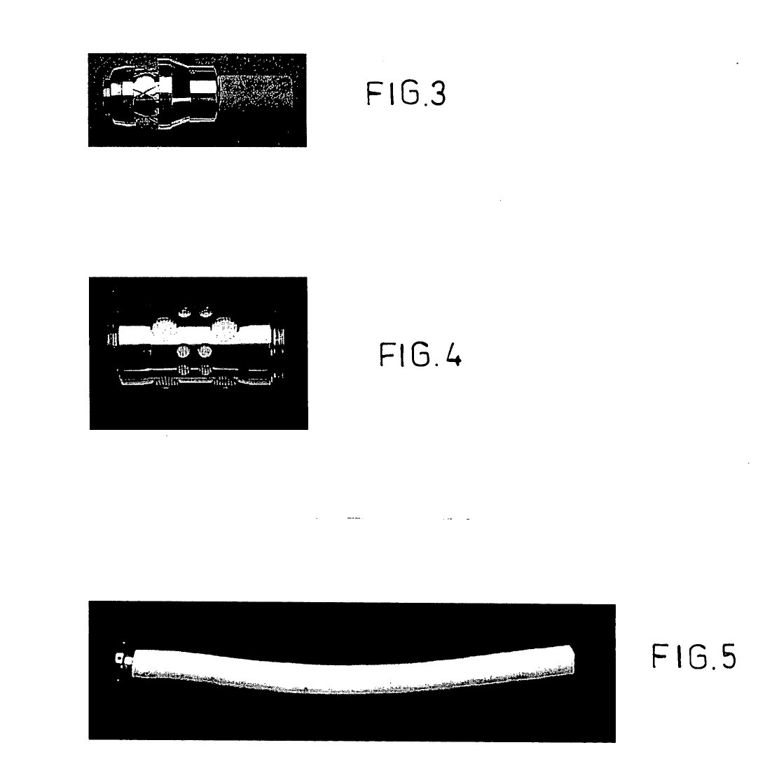

Accordingly, the assembled “HeartMate” device is shown in FIG. 2. A detailed view of the pump alone employed in the “HeartMate”device is shown by FIG. 3. In addition, a detailed view of the inflow conduit is shown in FIG. 4; and a detailed view of the outflow conduit is shown by FIG. 5. Each of these components is typically joined to a housing which directs blood flow to form the fully assembled device as seen in FIG. 2.

It will be noted and appreciated that, in a normal heart, blood moves form the right atrium to the right ventricle, which pumps it into the lungs to pick up oxygen. After receiving oxygen, the blood re-enters the heart by the left atrium; and then flows into the left ventricle, which pumps it with great force into the aorta to the body.

In a patient with congestive heart failure or a similar cardiac disorder, the surgically implanted VAD aids in the pumping of oxygenated blood for circulation throughout the body and serves as a left ventricular assist device (LVAD) in place of the weakened left ventricle for this purpose. In particular, the LVAD pumps blood in via an access tube from the left atrium/left ventricle and then pushes it through a conveying tube to the aorta, where the blood is then dispersed throughout the body.

Also, as shown by FIG. 1, an externally positioned battery pack provides electric power to the VAD. This was the original practice because the VAD was once powered by a small battery that needed to be charged every few hours and thus had to be kept outside of the body. This was extremely inconvenient. However, as time went on, the batteries became more high tech and longer lasting. Today, most batteries are positioned inside of the LVAD; nevertheless, they still must be periodically taken out to be recharged or replaced.

### II. Some Conventionally Available Ventricular Assist Devices (VADs)

A useful range and variety of ventricular assist devices and pumps are commercially manufactured today; are FDA approved for medical use; and are acceptable as devices and pumps suitable for surgical implantation into the chest of a living subject. Illustrative and representative examples of several are summarily described hereinafter.

There are a variety of different ventricular assist devices which can be used in-vivo as a bridge for heart transplant patients or as a bridge to recovery patients. A representative listing includes:

the HeartMate VE LVAD;

the Thoratec LVAD/RVAD/BVAD;

the Novacor LVAD;

the MicroMed (DeBakey) LVAD; and

the Arrow LionHeart VAD

the Levitronics VAD

the AbioMed device

the BioMedicus device

Several of these ventricular assist devices will now be summarily described in greater detail.

The HeartMate Pump

The HeartMate pump (illustrated by FIG. 2) is a pneumatically powered device that is implanted in the left upper quadrant of the abdomen. The pneumatic air hose exits from the lower half of the abdominal wall and is attached to a pneumatic power unit. This device is FDA approved and is now being used in a number of centers in the United States and abroad.

An electric version is available on an investigational basis and allows the patient to be considerably more mobile. With this system, a wire and vent tube pass through the lower half of the abdominal wall but, in place of the pneumatic drive unit, a shoulder holster with electric batteries is used to power the device. The HeartMate assist pump uses tissue valves while the flexible diaphragm is a textured surface. The patients are maintained on aspirin. This allows a thin, fibrin layer to develop on the blood contacting surface. The device has been generally free of thromboembolic complications. Blood can be taken only from the left ventricular apex and is pumped into the ascending aorta. The device cannot be used to access atrial blood, nor can it be used for right ventricular support.

The HeartMate assist pump uses tissue valves. The blood contacting component of the pump housing is lined with tiny titanium beads while the flexible diaphragm is a textured surface. After implantation, the recipient patients are maintained on aspirin. This pharmaceutical treatment allows a thin, fibrin layer to develop on the blood contacting surfaces of the pump.

The Thoratec Assist Pump

The Thoratec assist pump, manufactured by Thoratec Laboratories, Inc., is a pneumatically powered device that is typically placed on the anterior abdominal wall. The unit is versatile in that blood can be taken from the left atrium or the left ventricle and pumped into the aorta. Right heart support is readily provided by installing the pump to fill from the right atrium and pump blood to the pulmonary artery. The cannulas will pass through the chest wall in a manner similar to that of a conventional chest tube. A pneumatic power unit presently is used to provide the air pulses.

The Thoratec pump uses tilting disk type of mechanical valves and a highly smooth blood contacting surface. Patients in whom this device is employed are therefore maintained on low doses of either heparin or Coumadin.

The Novacor Ventricular Assist Pump

The Novacor ventricular assist pump is an electrically powered device built by the Novacor division of Baxter Laboratories. The pump is typically implanted in the left upper quadrant of the chest; and the electric line and vent tube are passed through the lower anterior abdominal wall.

The Novacor pump system uses biological valves and a highly smooth blood contacting sac. The Novacor mechanical design employs pusher plates on the front and the back of the sac, thus insuring excellent washout. Patients surgically implanted with this device are typically maintained on long-term anticoagulation drugs.

On a comparative basis, it is noteworthy that both the HeartMate electrical system and the Novacor unit were ultimately designed to be used as permanent ventricular assist devices. The opportunity to use these as bridge devices has provided the designers and the surgeons with an opportunity to gain experience with these pumps but without the concerns regarding long-term reliability (>one year). The electrical devices are designed to be implanted in the left upper quadrant of the abdomen, fill from the left ventricle, and eject into the aorta. Both of these systems require vent tubes and electric wires that exit from the lower abdominal skin. Ultimately, energy transfer by inductive coupling techniques can be employed with these two devices. However, some type of implantable compliance chamber would also be required to eliminate the need for any tube or wire to cross the skin.

The Micromed (DeBakey) VAD

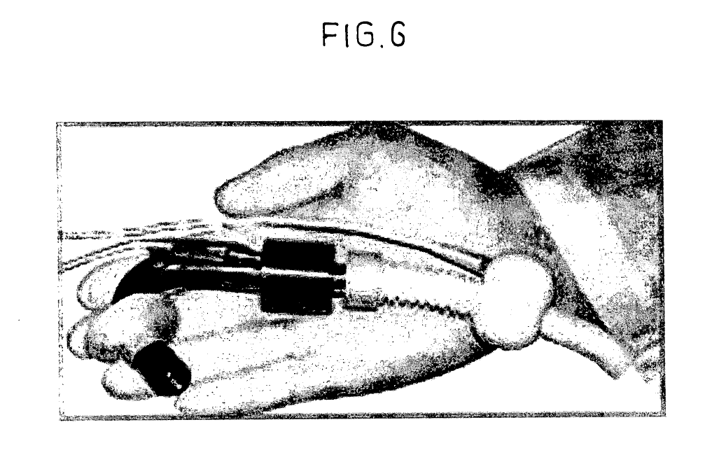

The Micromed (DeBakey) is an LVAD which is contained completely inside the body. This device is shown in FIG. 6.

As seen in FIG. 6, the DeBakey device is “non-pulsatile.” It moves blood continuously throughout the body instead of with the pump/relax cycle of a real heart. However, the non-pulsatile devices are nearly silent, while pulsatile devices can make quite a noise with each beat.

Currently, it takes less surgery time to implant a DeBakey VAD than other assist pumps (such as the HeartMate device)—about one and one half (1.5) hours. The DeBakey device is also currently less expensive. It has only one moving part: the inducer/impeller. Studies so far suggest that this VAD will last about 5 years before needing to be replaced.

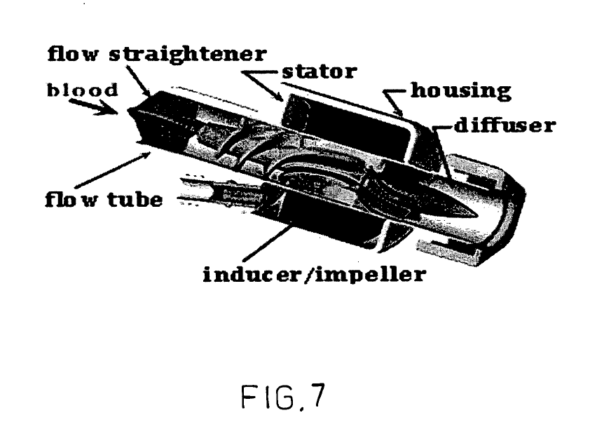

Within the DeBakey device, the inducer/impeller has 6 blades, with 8 magnets sealed in each blade. These features are shown by FIG. 7. As shown therein, the inducer/impeller of the DeBakey device spins between 8,000 and 12,000 times per minute; and this allows the device to pump up to 10 liters of blood per minute. All parts are enclosed in a sealed titanium tube. The pump is driven by a direct current motor stator with no brushes.

The titanium inlet tube of the DeBakey device is attached to the left ventricle; and the outlet tube is sewn to the aorta.

The main control unit of this pump is called the “Patient Home Support System,” This charges the batteries in the device and provides power when the person is stationary. The Patient Home Support System is the size of a small cooler and weighs about 10 pounds. The controller and 2 batteries fit into a carrying case with strap, which weighs about 5 pounds. The two batteries in this carrying case give a total battery time of 6 to 8 hours per charge.

The Arrow LionHeart Device

This Arrow LionHeart device is a “pulsatile” VAD; and uses a wireless power transmitter instead of wires going through the skin. The patient actually has a battery inside the body, allowing the patient to be completely disconnected from the external battery pack for as long as 20 minutes. Once the system is implanted in a patient, no wires or tubes go through the skin at all. The system uses transcutaneous, wireless power supply. One major advantage of this system is far less chance of infection.

The Arrow LionHeart is designed to be a permanent implant; and is manufactured in modules so parts can be changed without replacing the whole device. The VAD itself pumps fluid when a metal plate pushes on a plastic blood sac, forcing the blood out of the sac. The metal plate is driven by a miniature electric motor, with a controller that increases or decreases pumping as your activity level goes up and down. Also, there is no vent tube coming through the patient's skin (as with many other LVADs) because a compliance chamber in the VAD adjust for changes in gas volume with every beat of the pump. Air inside the compliance chamber helps in the pumping, but over time the air seeps through the synthetic wall of the chamber into the body and dissipates. The air has to be replaced once in awhile. New materials are currently being studied to fix this drawback.

### III. The Intended Uses And In-Vivo Applications For Ventricular Assist Devices (VADs)

The conventionally available ventricular assist devices (VADs, LVADs, RVADs, and BVADs) are of functional value and to be used with at least three (3) categories of people:

A. People waiting for a heart transplant. Many people who have such weak hearts will not live long enough to get a donor heart and under a complete heart transplantation procedure. These patients would therefore receive and accept a LVAD as a “bridge to transplant”. The implanted LVAD would stay in place until such time as when the donor heart transplant procedure is ready to be done; and then it is surgically removed of part of that subsequent procedure. Most patients in this group would receive an air-powered LVAD, which has a power supply the size of a desk, and which would require the LVAD patient to remain effectively stationary.

B. People who have had heart surgery but whose heart can't handle the circulatory load right after surgery. Doctors typically say these patients cannot be “weaned” from the heart-lung machine. As a Consequence, These patients can receive and medically accept a LVAD to keep them alive until their heart function returns to near-normal. This process usually only takes a few days

C. People who failed to respond to maximum drug therapy for their heart failure. The patients in this category would receive a portable electric LVAD on a permanent basis. Since the LVAD implant is permanent, the patients get to live at home, although there are restrictions as to what activities they can pursue.

### IV. The Surgical Method for Implanting a Ventricular Assist Device (VAD) into the Circulatory System of a Living Subject

The conventional surgical method for implanting a VAD in-vivo is a very complicated procedure, and must employ a heart-lung machine to keep the patient alive during the procedure. Also, the conventionally known procedure is also a very invasive procedure and is expensive to perform. The typical methods commonly used for implanting the conduits of a VAD include handsewn surgical anastomoses, where a surgeon places a series of surgical knots around the circumference of the vascular connection to form a liquid-tight connection; as well as a variety of vascular staple type devices, where mechanical apparatii are used to effect the connection. The latter generally use a two or more part apparatus comprising the staple introducer and an ‘anvil’ type of part against which the staples are curved back, bent, or otherwise fixed into position around the circumference of the vascular connection.

In contradistinction to conventional surgical practices, the present invention instead employs trocar based methods and apparatii for the creation of an end-to-end conduit (end-to-end between the conduit and pump, but side-to-end between the heart/blood vessel and conduit) connection using an implanted pump device to which inflow and outflow conduits have been attached; and uses a variety piercing introducer assemblies and systems for inserting this implantable VAD into the inside of the cardiac chambers and the blood vessel(s) of choice.

For the purposes of this description, the cardiac chamber and blood vessel of choice are individually punctured; and each receives the appropriate inflow conduit or outflow conduit extending from the pump device into its internal spatial volume. The receiving heart chamber and blood vessel serve as the source of blood to the pump and the recipient of blood flow from the pump respectively, depending on the conduit and the direction of blood flow.

A key advantage of the methods and devices described herein is the ability to attach and joi inflow and outflow conduits from the pump device while maintaining high blood pressures (systemic and greater) within the cardiac chambers or blood vessels. The VAD implantation procedure of the present invention can thus be performed without need to exclude blood from the aorta where the site of connection for the communicating channel or conduit is to be. This, in turn, obviates the need for use of the cardiopulmonary bypass machine, a device (which takes over the pumping of the blood through the body while the proximal aorta is made blood pressure free); and also eliminates any need for the Aortic Side Biting Clamp, a semicircular clamp which pinches off a portion of the aorta to create a blood pressure free pocket to which the handsewn conduit was previously made.

It has been long recognized that conventional use of both the heart/lung machine and the aortic side biting clamp result in trauma to the aorta; and such trauma causes the release of embolic debris from the aortic wall (a cause of stroke, cognitive deterioration, and other morbidities); as well as creates damage to the lining of the aorta (which can result in separation of the layers of the aorta) resulting in dissection, a potentially lethal complication. Frequently also, the time required for VAD implantation surgery is shortened because intricate in-vivo suturing techniques are no longer or minimally required to ensure that no leakage occurs at the anastomoses of the access or conveying conduits.

The Unique Surgical Method:

The present invention is concerned generally with minimally invasive methods for accessing the cardiac chambers and vascular system of the body; and is directed to an assembly and methodology for implanting inflow and outflow conduits between the cardiovascular system and a ventricular assist pump on-demand. In the surgical placement of VAD, it is necessary to attach outflow conduit or inflow conduit to different ports of the cardiovascular system. The tubular material used as the conduit may be either a synthetic or biological material. The inflow and outflow conduits are communication channels permanently joined to the cardiovascular structures as side (cardiovascular side) to end attachments, where the result is a tubular communication channel for continuous blood flow between the cardiovascular system and the pump.

It is useful here to understand in depth what the LVAD surgery entails and demands both for the patient and for the cardiac surgeon. In a LVAD operation, the improved surgical placement of a LVAD starts with preparation for cardiopulmonary bypass as follows:

The Initial Operational Steps:

The surgical placement of a LVAD in-vivo starts with preparation for cardiopulmonary bypass as follows:

1. Standard preparation and draping for cardiopulmonary bypass procedures is used. The groin is prepped and draped for femoral vascular access in patients with previous open heart surgery. Depending upon need for LVAD alone or for BVAD, one or two outflow conduits will need to be preclotted. These may be baked with albumin, or a more traditional approach using a mixture of patient blood and cryoprecipitate which is then sprayed with activated thrombin. Care is taken not to get material inside of the graft. Pumps are filled with 5% albumin to which 100 units of heparin/250 ml of albumin are added. Electrical and air connections of pump with are protected with the finger from surgical glove.

2. Cannulation for bypass consists of choosing a site high on the ascending aorta, often toward the inner curve of the arch. Choose an aortic cannula which allows the perfusionist to use cardiopulmonary bypass flows that are higher than normal if profound vasodilatation is seen. This has been known to occur in patients with severe congestive heart failure who have high levels of inflammatory mediators. The transesophageal echo is used to rule out the presence of a patent foramen ovale or atrial septal defect. If detected, bicaval cannulation is used for right atrial drainage, otherwise the two-staged venous cannula will be placed into the right atrial appendage. A Ferguson vent is placed via the right superior pulmonary vein into the left ventricle.

3. Full normothermic cardiopulmonary bypass is utilized. Cooling to 34 to 35 degrees C may help in maintaining vascular tone. Also, an aortic cross clamp is not necessary and, in fact, with an artrioventricular vent, cardioplegia is not required for myocardial preservation.

Choosing an Exit Site for the LVAD:

1. Once on full cardiopulmonary bypass with the lungs deflated the apex of the pericardium is fixed at least 3 cm above the left phrenic nerve with a tonsil and pulled medially. A 3-4 cm opening is created in the apex of the pericardium entering the left pleural space. With the tips of the index and middle fingers of the left hand the diaphragm is dissected down from the ribs in the costophrenic angle directly opposite the hole in the pericardium. Two finger breadths should be admitted. Feel for the tips of the fingers 3-4 cm below the left costal margin. A 1 to 1.5 cm skin button is excised, and using electrocautery the rectus muscle and its posterior sheath are opened over the fingers under the left coastal margin. Care is taken not to enter peritoneal space especially in patients with ascites.

2. A long Kelly clamp is passed through the tract from outside into the pericardium. The left ventricular (LV) apical (inflow) cannula with the end protected is then drawn through its tract. Approximately 3 cm of the Dacron velour covered portion should protrude out of the skin and the apical fixation felt sewing cuff should be flush with the pericardium. The pump is brought to the field and the inflow connector is aligned with the cannula in order to determine the location of the skin button for the inflow conduit. A skin button is made, similar to the previous button, and the hole is extended through the rectus muscle and its posterior sheath. The inflow conduit is then pulled through this new tract in a direction from pericardium to the skin. Again ensure 3 cm of Dacron covered cannula extends beyond the skin opening. Size the graft to length measuring enough in its extended position that it reaches the right lateral border of the ascending aorta with redundancy to allow graft to lie under the right half of the sternum. Cut graft with 45 degree angle. Remove inflow and outflow conduits from their tracts.

Aortic Anastomosis:

Place a partial aortic clamp (Setinsky) on ascending aorta and make linear aortotomy. The graft is sewn end-to-side on aorta with 4-0 Proline suture. The partial clamp is removed from aorta and graft is clamped with a soft clamp (Fogarty). If a BVAD is required, then proceed with the next step; otherwise, proceed to Left Ventricular Apical Cannulation.

Choosing an Exit Site for a RVAD:

Place curved portion of atrial cannula next to right atrial appendage and approximate skin exit site under right costal margin. Exit site should be approximately 3 cm below costal margin and in mid-clavicular line. Make a skin button as for the RVAD. Just as in the LVAD, the inflow cannula exit site, because it is in a fixed position, will determine the exit site for the RVAD outflow conduit and should be made first. Pass the inflow conduit through the skin opening and into the pericardium and let it remain next to the right atrium. Again, bring RVAD to the field and align inflow cannula with its connector to determine skin button site for inflow conduit. Make skin button and tract under rectus muscle as for LVAD inflow graft. Pass the graft through the tract and cut to a length to pass easily over the right ventricle (RV) to the pulmonary artery. Remove the pulmonary artery inflow graft for later use. The RVAD inflow graft will ultimately lie over the LVAD outflow graft.

Pulmonary Artery Anastomosis:

Place two silk sutures on the pulmonary artery (hereinafter “PA”). The first is located at the pulmonary artery bifurcation; and the second is placed at the level of the pulmonary valve. Raise up the PA with these tagged sutures. A linear pulmonary arteriotomy is made. A Ferguson vent is passed through the RVAD outflow graft into the PA to allow an anastomosis to be made using a 4-0 Proline suture, without clamping the PA. Clamp the PA graft after removing the vent.

Left Ventricular Apical Cannulation:

The heart is elevated out of pericardium with laparotomy pads. An apical dimple is easily felt 2 cm lateral to the left anterior descending coronary artery. The apical cannula is placed against the LV apex and 8 to 12, 3-0 pledgetted Tevdek sutures are passed radially full-thickness through the apical LV muscle and then through the felt sewing cuff of the apical conduit. Sutures are placed on snaps. Apical core is made by incising the muscle with a #11 scalpel blade. The first incision should start on the side away from the septum to avoid septal injury. Core should be no larger than 1.5 cm. Check for thrombus in LV and excise excess trabeculae. Hole should be firm enough so that mild resistance is felt as LV apical cannula is screwed into the LV apex. Apical sutures are tied firmly to buttress LV muscle under felt sewing cuff.

Connecting the LVAD to the Cannulae:

1. Pass a Kelly clamp back through the tract for the apical (inflow) cannula (lateral most tract). While supporting the heart with right hand the left hand is used to bend the apical cannula near the apex. An assistant grabs end of cannula with the Kelly clamp and pulls it through tract while surgeon slowly lets cannula straighten and eases the heart down into pericardial space. Aortic inflow cannula is passed through its tract. Make sure that both cannulae have been pulled through their respective tracts to the position that will be desired when the sternum is closed. It is essential that the ventricular apical cannula be pulled down enough so that the apex of the heart is flush with the pericardium.

2. The LVAD cannulae are trimmed to a length which allows best positioning of the pump unit. If patient has been cooled start rewarming during this phase. Approximately 4 cm of non-Dacron velour covered cannula must be left to allow for easy placement of the cannula on the connector of the pump. Connect the apical conduit to LVAD. Pass the pulmonary artery conduit through its tract and pull it through the skin. Make sure this conduit passes anterior to the aortic conduit. Place a de-airing access hole in aortic graft and pass the de-airing catheter back through graft and across the outflow valve of the LVAD. A Swan-Ganz catheter or a 5 to 7 F Cordis angiography catheter can be used for this purpose. Connect the outflow conduit to the outflow connector of the LVAD. When connecting the LVAD to its conduits, use the appropriate collet and collet nut or else the conduits may detach while pumping. The VAD is supplied with a white nut and smaller collet on the outflow side. This is the same for either an LVAD or an RVAD. On the inflow side it comes with a larger collet and a black collet nut. This is intended for RVAD or atrial cannula use only. For the LV apical conduit you need to exchange the black nut and larger collet for a white nut and smaller collet supplied with the apical cannula.

De-Airing the LVAD:

Ensure that the patient has been warmed, inflate the lungs and begin normal ventilation, start appropriate inotropic support for right ventricle if indicated. Continuous assessment for ventricular and aortic air with transesophageal echocardiography is essential during the de-airing process. Connect the air drive line to the LVAD first since this requires the most force, then connect the electrical lead. Place the patient in steep Trendelenburg position and check left ventricle for gross air after reducing pump flow by enough to allow heart to fill gently. Remove gross air using superior pulmonary artery vent; then remove the vent. Bring the flow of cardiopulmonary bypass down to two liters/minute and begin to remove air from the pump sac by drawing on the de-airing catheter with a 30 cc syringe with a stop-cock. If the tip of catheter is visualized in pump most of the air can be removed by tilting the pump to direct bubbles to tip of catheter. Occasionally unclamp the aortic graft to allow air trapped in the conduit to exit via the de-airing site in graft. Single cycles of the pump are used to expel any air from the ventricle. Remove the clamp on aortic graft and while visualizing with trans-esophageal echocardiogram (TEE) the aortic root a single pump cycle is run to assess for air in the device. If air removal is judged adequate, remove the de-airing catheter and begin the LVAD at a fixed rate of 40 beats per minute. If an RVAD is also to be used, trim the outflow cannula to the desired length. DO NOT CUT THE ATRIAL CANNULA.

Connect the RVAD to the pulmonary artery conduit. Connect the air and electrical leads for the RVAD. Gradually discontinue cardiopulmonary bypass and allow the LVAD to run in “fill-to-empty” mode. Check for adequacy of LV drainage with TEE.

Completion of RVAD Insertion:

After coming off bypass, the right ventricle (RV) will need support with inotropic agents. Remove the clamp from PA graft and allow back-bleeding to partially fill the RVAD unit. Remove the right atrial cardiopulmonary bypass venous cannula and insert RVAD atrial cannula. Allow back-bleeding of atrial cannula to fill the rest of the RVAD, connect the atrial cannula, secure it to the connector and begin RVAD pumping in “fill-to-empty” mode. De-airing is usually satisfactory using this method and the de-airing catheter is not required.

Completing the Surgery:

Reverse anticoagulation completely and when finished transfusing patient blood decannulate. An alternative way of protecting the grafts is to longitudinally split a Gore-Tex graft and place this over the Dacron grafts of the VAD. The mediastinum is irrigated with large amounts of saline solution with both antibiotics for Staphylococcus, and Amphotericin to reduce fungal infection.

Preparation of the Pump:

The pump is assembled and calibrated on the back table by an assistant, as detailed in the instruction manual. This includes:

a. Preclotting valved inflow and outflow conduits with the patient's non-heparinized blood.

b. Baking the outflow graft in albumin or plasma.

c. Filling the pump with saline solution.

Preperitoneal Pocket Construction:

1. A midline incision is made extending from the sternal notch to the umbilicus with division of the linea alba. 2. Sternotomy is performed prior to creation of the pocket in order to have quick access to the heart in the event of hemodynamic instability.

3. The preperitoneal fat is dissected from the undersurface of the rectus sheath using low power cautery. Superiorly the dissection is carried to the undersurface of the diaphragm until the apex of the heart can be palpated just lateral to the inferior phrenic artery and vein. These vessels should be ligated to avoid injury during transdiaphragmatic placement of the inflow cannula, as bleeding from this site is difficult to visualize after device insertion. If the peritoneum is entered during the dissection, the defect is repaired with prolene sutures to prevent herniation of abdominal contents. If the desired plane is difficult to develop, the rectus sheath can be entered and the posterior sheath left as a patch overlying the peritoneum.

4. This dissection must be carried well back into the retroperitoneum (posterior to the spleen) to allow adequate mobilization to fit the device preperitoneally

5. A plastic model of the device can be inserted into the pocket to assess whether enough room is available for the device.

6. The preperitoneal space to the right of the linea alba is also opened for about 2 to 3 cm to facilitate closure of the linea alba at the completion of the case and allow room for the device outflow valve and graft conduit.

7. The muscular attachment of the right hemidiaphragm to the medial edge of the sternum must also be divided to allow room for the graft.

Driveline Tunnel Creation:

1. The driveline usually exits in the right lower quadrant of the abdomen, approximately at McBurney's point.

2. A small incision is made (approx. 85% of the driveline diameter), and a tunneling device passed subcutaneously inferiorly around the umbilicus, and then into the pocket through the rectus sheath at it's most inferior aspect. This entry point into the pocket should later be examined to make sure there is no bleeding from the rectus muscle or an arterial branch. The tunneler is then screwed onto the end of the driveline, which is then pulled back through the tunnel to the skin.

3. The drive line is not sutured or otherwise attached to the skin.

Attaching the Apical Cuff and Transdiaphragmatic Passage of the Inflow Cannula:

1. A standard dose of Heparin is given and cardiopulmonary bypass instituted using standard aortic and dual stage venous cannulae. The aorta is not crossclamped although it can be.

2. The apex of the left ventricle is elevated.

3. There are two approaches that can be used at this juncture: 1) the apical cuff sutures are placed first and then the myocardium cored out or 2) the hole is first made with the coring device and then the apical cuff sutures placed.

a. An apical vent is passed into the left ventricle. The apical vent will serve as the center of the sight of insertion of the apical cuff. Pledgeted 2-0 ethibond sutures are placed circumferentially partial thickness into the myocardium then passed through the sewing ring of the apical cuff. The coring device is then used to cut a hole in the myocardium and the sutures are secured.

b. If necessary the heart can be vented through the right superior pulmonary vein. The coring device is used to make the hole in the apex. This technique is particularly useful if there is ventricular thrombus or the myocardium is friable from recent infarction.

4. To core the apical hole the ventricle is distended and the coring knife is aimed towards the lateral wall to avoid entering or positioning the inflow cannula towards the septum. Residual muscle or scar that may impinge on the cannula site is resected. A search for loose mural thrombus is made, adherent thrombus is left in place.

5. For either method it is important to ensure that more myocardium is gathered at the perimeter of the sewing circle than at its center. These sutures are deep but not full thickness. If a coronary vessel is lacerated, the next suture should incorporate the bleeding site, as it is very difficult to visualize this area once the device has been attached.

6. Once the apical cuff is secure, a cruciate incision in the diaphragm opposite the ventricular apex is made just lateral to the inferior phrenic vessels, and the device inflow cannula is brought into the chest. The inflow cannula is then inserted through the apical cuff until the entire titanium surface is within the cuff. The surgeon should aim the cannula away from the interventricular septum to prevent inflow restriction aspiration of the ventricular septal muscle into the cannula. The dacron tie of the inflow cuff is then secured and an additional plastic band and dacron tie used to reinforce the connection and flatten out the silicone cuff to minimize the risk of aspirating air into the device. Blood is now allowed to passively fill the device and exit via the outflow valve, serving as a vent and de-airing the device.

Anastamosis of the Outflow Graft to the Ascending Aorta:

1. A partial occluding clamp is placed on the right lateral aspect of the ascending aorta and a longitudinal aortotomy performed. The periaortic adventitia is left in place, and a strip of bovine or native pericardium incorporated into the anastomosis.

2. The outflow graft is usually cut to a length of 12 to 15 cm. If too long the graft will kink as the chest is closed. Finally the connector from the outflow graft is inspected for thrombus and cleaned.

3. If the outflow graft is not attached to the housing of the pump already, make sure the nut to secure it has been placed on the graft prior to anastamosis to the aorta.

4. The anastamosis is created with 4-0 Prolene suture. The apex and heel of the anastamosis are reinforced with interrupted 4-0 prolene pledgeted horizontal mattress stitches. These are common sites of postoperative bleeding.

5. The outflow graft connector should be clean and free of thrombus. Thrombus can interfere with the creation of a water-tight seal and lead to significant hemorrhage. Bleeding from this connector can be treated by wrapping the entire connector circumferentially with a strip of bovine pericardium, and securing it at multiple sites with heavy silk ties to tamponade the leak.

De-Airing the Pump:

1. Large pockets of air are retained in the pump during implant and must be removed prior to initiating complete support.

2. Components of the de-airing process include:

a. Placing the patient in steep Trendelenburg position

b. Volume loading, ventilation and the reduction of CPB flow to move air from the lungs and pump to site of egress.

c. Sights for air to escape include

d. a purposely loose outflow graft connection

e. an 18 gauge needle placed in the outflow graft at its highest point

f. a 14 gauge ascending aortic root cannula placed to suction

3. A vascular clamp remains on the outflow graft distally. The pump is hand cranked. The housing of the pump is shaken repeatedly to release air. de-airing is continued until no air is seen by TEE and no air exits through the de-airing sites on the outflow graft.

4. The outflow connector is tightly screwed together and secured with the heavy ethibond suture supplied with the Heartmate. The 18 gauge needle is removed from the graft and its insertion site closed with a 4-0 prolene.

Starting the Pump and Weaning from Cardiopulmonary Bypass:

1. The field is flooded with saline to prevent aspiration of air through the inflow valved conduit and connector in the case of inadequate pump filling.

2. Inotropic support is started before separating from bypass and activating the LVAD. Dobutamine and Milrinone are useful inotropes particularly in the presence of pulmonary hypertension, norepinephrine and vasopressor are added to maintain a mean blood pressure greater than 65 mmHg.

3. Bypass flow is decreased to 2 L/min, the heart filled with volume and the device started in the fixed mode at 20 cycles per minute. If filling is adequate the rate is increased as cardiopulmonary bypass flow is reduced. The device switched to automatic mode after the cessation of cardiopulmonary bypass.

4. Transesophageal echocardiography is used to ensure adequate ventricular decompression with bowing of the septum away from the right ventricle. A bubble study is also performed to rule out a patent foramen ovale, which can result in severe hypoxemia due to a right to left shunt.

5. A thermodilution cardiac output is performed and compared to the output from the LVAD. A difference of greater 20% between the right heart output and the measured device output signifies significant aortic regurgitation, which will need to be addressed, by either sewing the noncoronary and right coronary valve leaflets together or oversewing the valve completely.

6. Protamine is given and clotting factors used as needed. In the presence of severe coagulopathy the chest may need to be packed open.

Preferences:

Devices:

Heartmate™ LVAS (Left Ventricular Assist System)

Heartmate™ VE LVAS (Vented, Electrically powered Left Ventricular Assist System) Thermocardiosystems Inc.470 Wildwood St. Woburn, Mass. 01888, 1-781-932-8668

Bovine pericardium

Instruments:

Standard Cardiac Instrument Tray

Other necessary instruments are included with the device

Sutures:

Ethibond 2-0 with pledgets

Prolene 4-0 with and without pledgets

Tips and Pitfalls:

This device currently favors preperitoneal placement of the LVAD. Significant morbidity including colonic perforation, small bowel obstruction, diaphragmatic hernia, and wound dehiscence were encountered during the initial experience with an intra-abdominally placed LVAD.

To decrease the incidence of driveline infections, the driveline tunnel is made as long as possible.

Transesophageal echocardiography is a useful adjunct during insertion. It can be used to determine the source of hemodynamic problems after insertion (e.g. the presence of a PFO, obstruction of the inflow cannula by the septum).

Aprotinin is used routinely for both the implant and the explant/tranplant operation. If the patient has previously been exposed to aprotinin it is not infused until the patient is prepared for the initiation of cardiopulmonary bypass, should an allergic reaction and subsequent cardiovascular collapse occur.

### V. Embodiments of the Piercing Introducer Assembly Useful for Juncture of the Inflow and Outflow Conduits of a Ventricular Assist Device

The piercing introducer assembly intended for use with the present surgical method was previously disclosed in full as the inventive subject matter of U.S. Pat. No. 6,669,708 issued Dec. 30, 2003, the entirety of which is expressly incorporated by reference herein.

### A. One Preferred Format

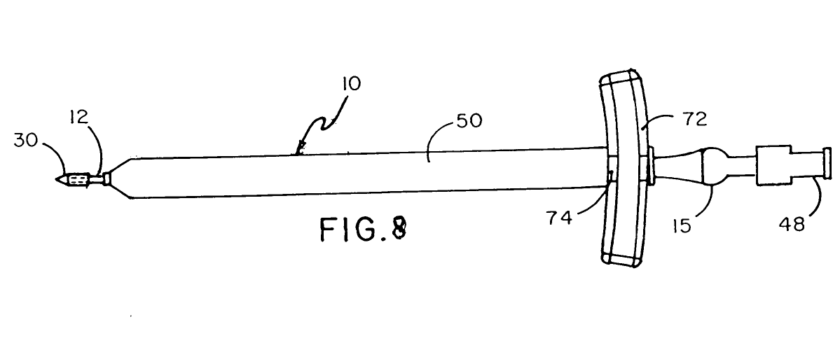

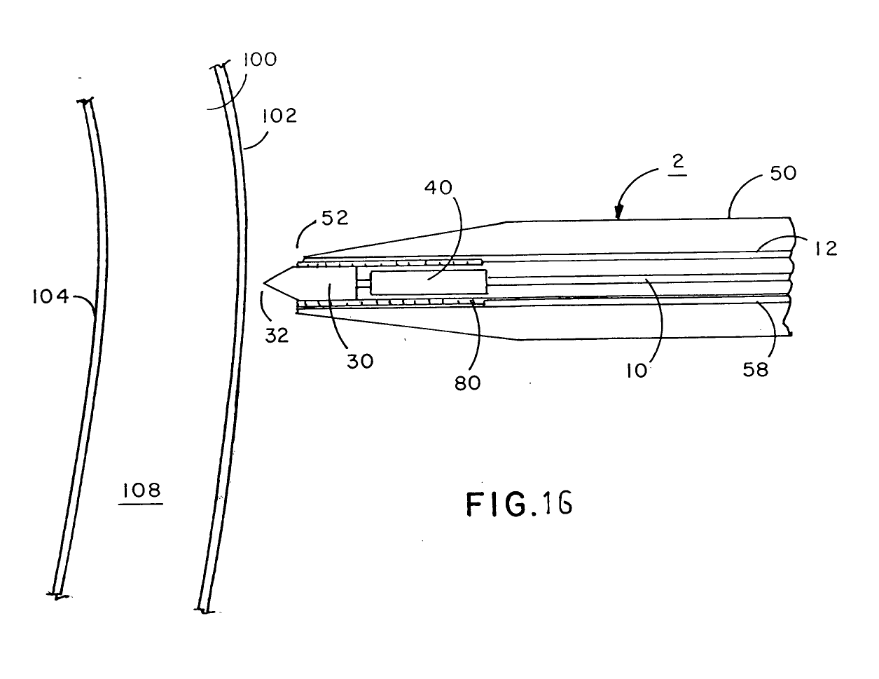

A preferred format and embodiment of the piercing introducer assembly is exemplified and illustrated by FIGS. 8-21 respectively. As shown therein, FIGS. 8-15 identify the preferred introducer assembly in its minimal and optional component parts; while FIGS. 16-21 respectively illustrate the intended method of using the introducer assembly to achieve a sutureless juncture of a prepared communication channel or tubular conduit to either a blood vessel or to the interior of a cardiac chamber (typically a ventricle of the heart).

The introducer assembly as a whole is illustrated by FIG. 8. As seen therein, the optimized introducer assembly is comprised of a perforator instrument 10; and the communication channel or tubular conduit controlling means 40, which appears as an inflatable and deflatable on-demand balloon appliance in this preferred embodiment; a volumetric sheath 50; and sheath position holding means which appear in this preferred embodiment as the grasping member 70.

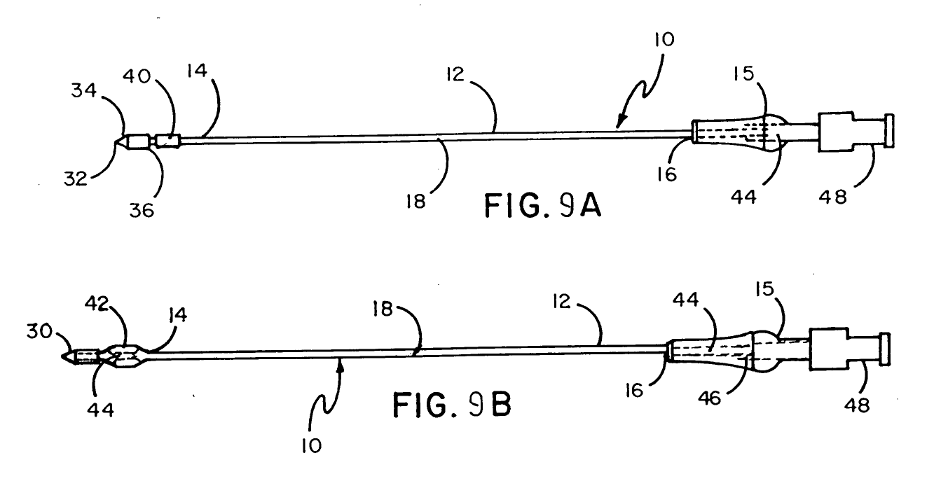

The introducer assembly exemplified by FIG. 8 is in completely assembled form; comprises each of the requisite and optional component parts and sub-assemblies in its appropriate placement and position; and shows the entire optimized apparatus in a state ready for immediate usage. Details of the individual component parts of the introducer assembly are shown by FIGS. 9-15 respectively.

FIG. 9 shows the minimal introducer assembly in detail which comprises only the perforator instrument 10 and the balloon appliance 40 which serves as one specific means for controlling and deploying a prepared communication or access conduit. As illustrated by FIGS. 9A and 9B, the perforator instrument 10 of the minimal introducer assembly is comprised of at least one elongated supporting shaft 12 of predetermined overall dimensions and axial length having two ends 14, 16; and has an internal lumen 18. knob handle 15 is attached at the end 16 of the supporting shaft 12; and a perforating headpiece 30 is joined to the supporting shaft at the other shaft end 14. The perforating headpiece 30 is integrally joined to the end 14 of the supporting shaft 12 and itself comprises a perforating tip 32, a penetrating body 34, and a base aspect 36. The perforator instrument 10 is thus itself an assembly of parts which provides a knob handle for the surgeon and a cutting headpiece suitable for penetrating the sidewall tissue of a blood vessel or cardiac chamber and forming an aperture in-situ.

Disposed adjacent to the perforating headpiece 30 on the supporting shaft 12 of the perforator instrument 10 is an inflatable and deflatable on-demand balloon appliance 40. In this minimalist format and first preferred embodiment, the balloon appliance 40 structurally serves as communication or access conduit controlling means for the deployment of the introducer assembly as a whole; and provides the primary apparatus for controlling the positioning of a previously prepared communication or access conduit, which, after proper placement within the assembly, will serve either as a communication or access conduit.

The balloon appliance 40—the communication or access conduit controlling means in this embodiment—is comprised of an expandable and deflatable balloon 42 whose interior volumetric space can be increased and decreased on demand repeatedly without difficulty; an inflation line 44 joined to the interior space of the balloon 42; and a luer lock fitting 48 joined to the inflation line 44 but positioned adjacent to the knob handle 15. The luer lock fitting 48 provides the direct communication means for introducing a inflation fluid from an external source (not shown) into the inflation line 44 through which the inflation fluid will be carried and transported into the interior volumetric space of the balloon 42. By adding fluid through or allowing fluid to flow out of the luer lock fitting 48, the degree of inflation or deflation for the balloon appliance 40 can be controlled and maintained at will.

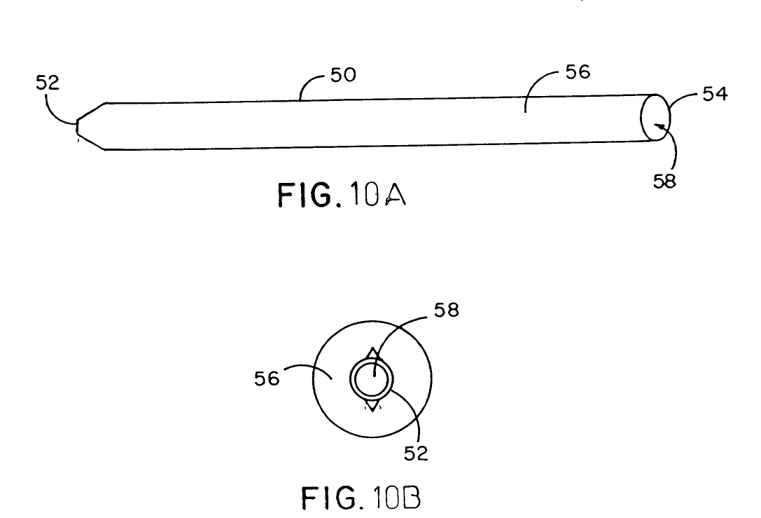

The volumetric sheath 50, an optional but highly desirable structure of the introducer assembly, is illustrated by FIGS. 10A and 10B respectively. The optional volumetric sheath 50 has two open ends 52, 54 and at least one sidewall 56 of predetermined dimensions. The volumetric sheath 50 is sized at the open end 52 for on-demand placement adjacent to and aligned closure with the perforating headpiece 30 of the perforator instrument 10. In addition, the optional volumetric sheath 50 is substantially annular in configuration over its axial length but is desirably constricted at the open end 52 to conform to the particular dimensions of the perforating headpiece 30. The essential purpose and function of the volumetric sheath 50 is protection such that its internal spatial volume 58 over its axial length becomes available and adapted for protective positioning around and volumetric spatial envelopment of at least a portion of the supporting shaft 12 which extends from the perforating headpiece 30 of the perforator instrument 10.

As shown in FIG. 8 previously, the optional volumetric sheath 50 when properly positioned provides a protective covering and envelope for the spatial volume and ambient environment then surrounding the supporting shaft 12; and any contents (including a prepared communication or access conduit which is then positioned within the internal spatial volume 58 of the volumetric sheath 50) will become protectively surrounded and enveloped by the sheath sidewall 56 over the entirety of the axial length for the configured volumetric sheath 50. For the introducer assembly as a whole, particularly as depicted by FIG. 8, the volumetric sheath 50 provides the protective envelopment of an ambient environment spatial volume and all its interior contents which then surround the supporting shaft 12 and the introducer assembly as an integrated unit.

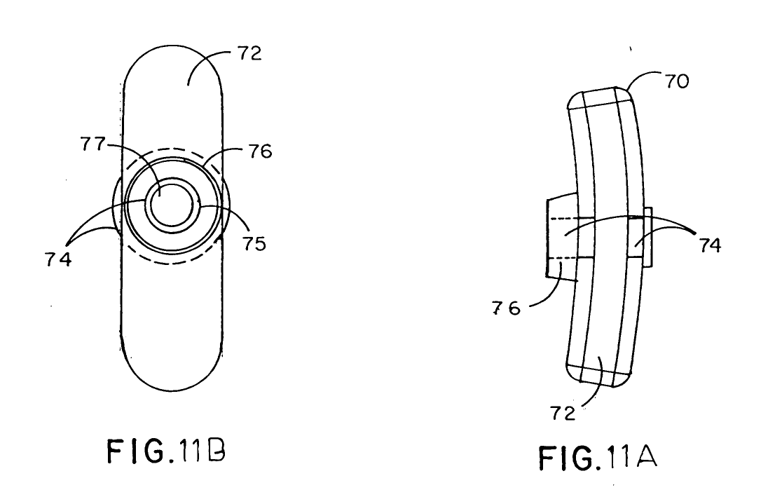

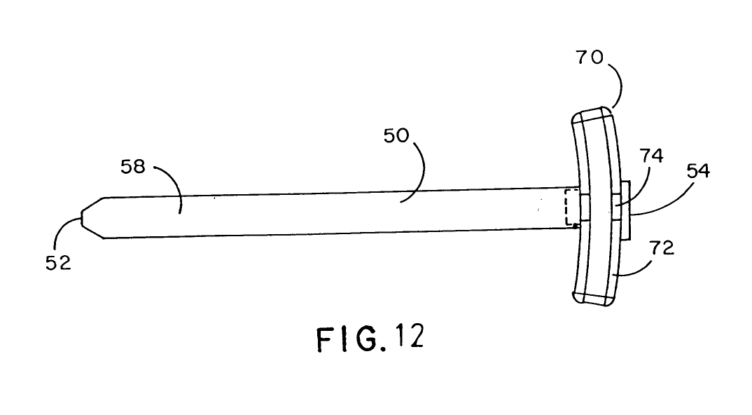

The optional position holding means 70 and its intended function within the preferred introducer assembly is illustrated by FIGS. 11 and 12 respectively. FIGS. 11A and 11B each illustrate the grasping member 70 which is the specific embodiment of the optional position holding means in this assembly; while FIG. 12 shows the interrelationship between the grasping member 70 and the volumetric sheath 50 as intended by the assembly of parts.

As shown by FIGS. 11A and 11B, the grasping member 70 comprises a grip 72; a shaft mounting 74 configured for disposition around the support shaft 12 of the perforator instrument 10; and a sheath positional end fitting 76 which is annular or circular in overall configuration and dimensioned to fit snugly in a friction holding position with the open end 54 of the volumetric sheath 50. It will be noted and appreciated also that the shaft mounting 74 is itself substantially circular in configuration and is comprised of a flange 75 and a encircled aperture 77 through which the supporting shaft 12 will pass axially.

When properly aligned with the optional volumetric sheath 50, the overall result is illustrated by FIG. 12. Clearly, the open ends 52, 54 of the volumetric sheath 50 are in alignment with the grasping member 70; and the entire internal spatial volume 58 of the volumetric sheath 50 is encompassed by the attachment of the position holding grasping member 70 at the end 54. The grasping member thus provides position holding means and maintenance for the volumetric sheath within the introducer assembly over most of its axial length.

The arrangement of each of the requisite and optional component parts illustrated by FIGS. 9-12 is thus shown properly aligned and assembled as a preferred structural apparatus by FIG. 8. As the grasping member 70 is advanced forward or pulled rearward over the supporting shaft 12 of the perforating instrument 10, the volumetric sheath 50 will concomitantly be advanced forward or pulled rearward as a consequence. Thus, at any moment or instance of use, the volumetric sheath 50 as a whole and its internal spatial volume 58 as well as any contents to be found within the internal spatial volume itself can be advanced to and beyond the perforating headpiece 30 or pulled rearward to reveal the component parts of the perforator instrument. In this manner the perforating headpiece 30 can be alternatively and repeatedly exposed or hidden within the internal spatial volume 58 of the volumetric sheath 50.

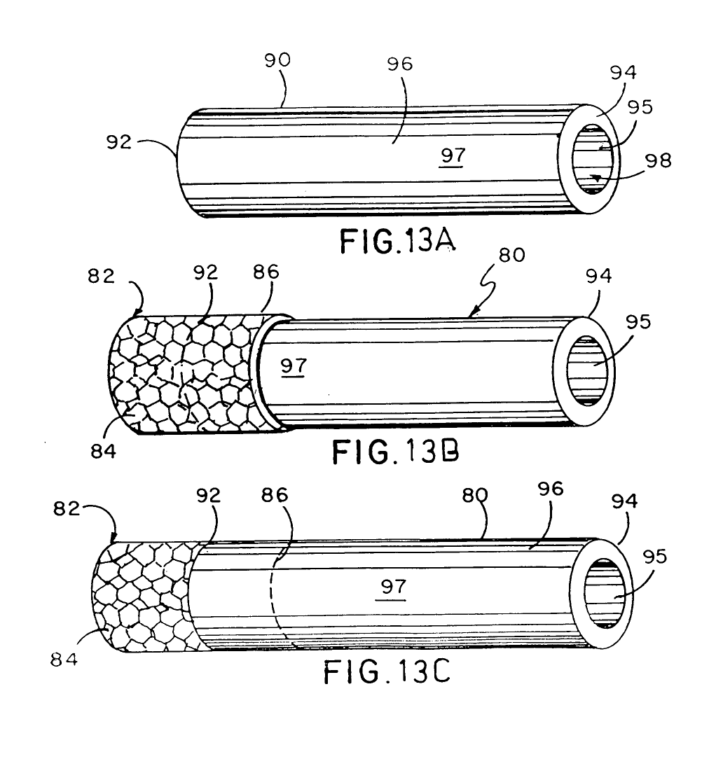

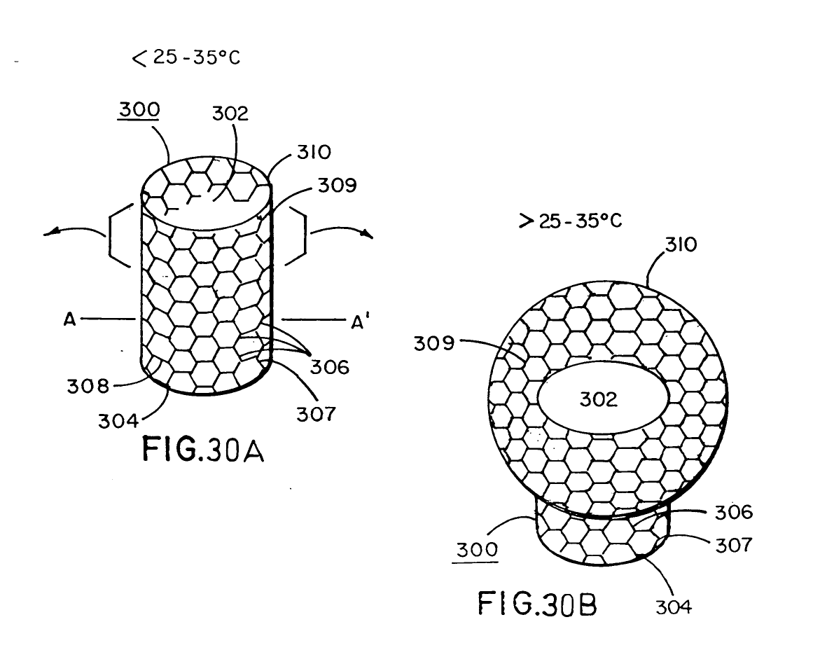

The purpose and function of the piercing introducer assembly is to provide for a catheterless and sutureless juncture of a prepared communication or access conduit to the interior of a blood vessel or a cardiac chamber in-vivo. For descriptive purposes, the prepared communication channel or access tube is illustrated by FIGS. 13A, 13B, and 13C, which show the parts of a properly prepared conduit to be used subsequently as either an inflow or outflow conduit. The essential parts are briefly illustrated by FIG. 13; but a far more detailed description of the major forms and alternative embodiments of such communicating conduits as a prepared article are subsequently disclosed herein as well as illustrated by FIGS. 30-43 inclusive.

As shown by FIG. 13, a prepared communication channel 80 suitable for access and conveyance of fluid blood in-vivo is comprised of a linking connector 82 and a tubular conduit 90. The tubular conduit 90 is any tube or hollow conduit having two open discrete ends 92, 94; at least one tubular sidewall 96; and an internal lumen 98 of fixed spatial volume. The tubular conduit 90 accordingly also has an internal sidewall surface 95 which is co-extensive with the internal lumen 98; and an external sidewall surface 97 of predetermined dimensions and overall configuration. Further details regarding the tubular conduit 90 are described hereinafter.

The linking connector 82 is shown as an open wire meshwork construction in FIGS. 13B and 13C respectively. The linking connector includes at least a first cuff portion 84 of predetermined dimensions and configuration which is superelastic and/or thermo-elastic, thermo-plastic and deployable on-demand. The first cuff portion 84 is configured for passage through an aperture in the wall of a blood vessel or a cardiac chamber; is superelastic or thermo-elastic; and is deformable and deployable on-demand whereby the act of deformation in-situ within the interior volumetric space of a blood vessel or cardiac chamber serves to secure the joined tubular conduit interior of the blood vessel or cardiac chamber and places the secured tubular conduit in fluid flow communication with the interior volumetric space of the blood vessel or cardiac chamber proper. The linking connector also includes a second conduit retaining portion 86 of determined dimensions and configuration which is joined to the sidewall 96 of the tubular conduit 90 such that the joining retains and secures the tubular conduit 90 for fluid flow communication purposes.

The juncture of the linking connector 82 may be made either at the external sidewall surface 97 as shown in FIG. 13B or alternatively at the internal sidewall surface 95 as illustrated by FIG. 13C. In many instances the juncture of the second conduit retaining portion 86 is desirably done within the internal lumen 98 by direct joining to the internal sidewall surface 95. However, any format of juncture [using staples, sutures or any other permanent means for joining] is suitable for use within the introducer assembly. Accordingly, the prepared communication channel 80 as a prepared article of manufacture is shown equally by FIGS. 13B or 13C without distinction or meaningful difference.

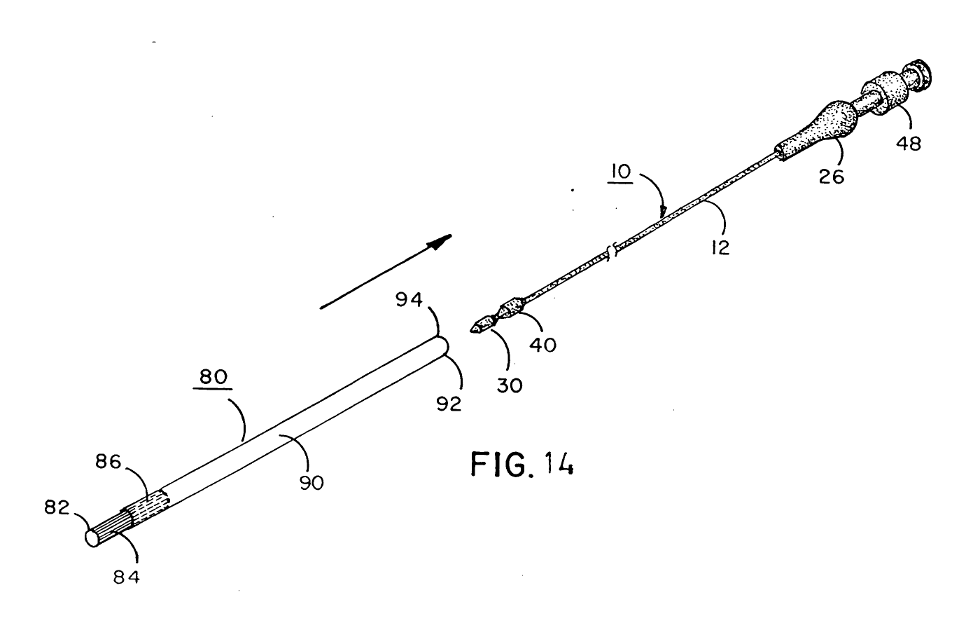

For purposes of further description the communication channel 80 will be prepared in the manner illustrated by FIG. 13C where the linking connector 82 is joined along its retaining portion 86 to the internal sidewall surface 95 of the tubular conduit 90. The intended placement of the prepared communication channel 80 as embodied by FIG. 13C is shownin FIG. 14.

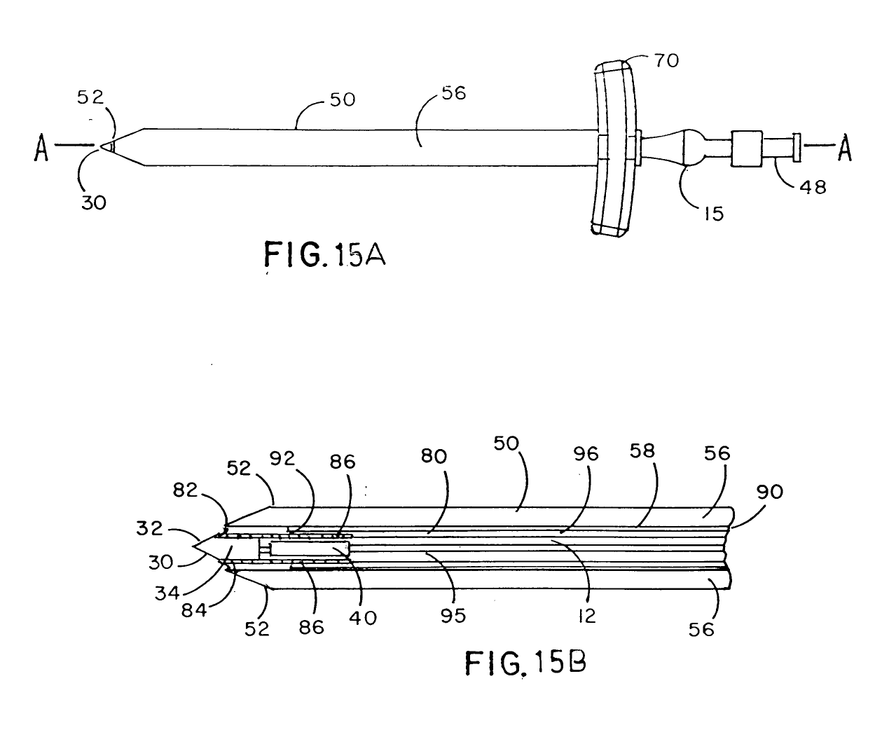

As illustrated by FIG. 14, the prepared communication channel 80 is intended to be positioned over perforator instrument 10. This positioning is accomplished by inserting the perforating headpiece 30 and the supporting shaft 12 of the perforator instrument 10 into the internal lumen 98 of the tubular conduit 90 via the open end 94. The perforating headpiece 30 is then extended through the internal lumen 98 until it exits the communication or access conduit 80 at the other tubular conduit end, thereby concomitantly also passing through the joined linking connector 82 in its entirety. Supporting shaft 12 will then hold and support the entirety of the prepared communication or access conduit 80 in this position within the introducer assembly; and the volumetric sheath with grasping member 70 is subsequently placed around prepared communication or access conduit 80. This results in the completely arranged introducer assembly illustrated by FIG. 15.

As seen therein, FIG. 15A shows a perspective view of the complete introducer assembly with the prepared communication channel 80 contained within the internal spatial volume 58 of the volumetric sheath 50. To illustrate better the aligned positioning within the introducer assembly, a cross sectional view along the axis AA′ of FIG. 15A is provided and shown in detail via FIG. 15B. As seen therein, the prepared communication channel 80 is housed within the internal spatial volume 58 of the volumetric sheath 50; is completely enveloped by the volumetric sheath 50; and is protected by the covering of the volumetric sheath 50 while supported on the supporting shaft 12 of the perforator instrument 10. The first cuff portion 84 has been placed adjacent the penetrating body 34 of the perforating headpiece 30 while the second conduit retaining portion 86 joined to the internal sidewall surface 95 of the tubular conduit 90 appears positioned around the balloon appliance 40. As noted previously, the balloon appliance may be inflated and deflated at will; and by inflating the balloon appliance 40 in this setting, the inflated balloon will thus hold the entirety of the prepared communication channel 80 firmly and indefinitely and prevent the conduit from moving linearly until such time that the balloon 40 is deflated again. Equally important, the entirety of the perforator instrument 10 including the perforating headpiece 30 may be advanced forward or pulled rearward at will at any time while positioned within the internal lumen 98 of the tubular conduit 90 and the joined linking connector 82. In this manner, the entire axial length of the perforator instrument may be advanced or withdrawn while the prepared communication channel 80 remains in a single position within the enveloped spatial volume 58 provided by the protective volumetric sheath 50.

The complete introducer assembly illustrated by FIG. 15 is shown in the intended application and usage for the introduction and sutureless juncture of a prepared inflow or outflow conduit by FIGS. 16-21 respectively. These FIGS. 16-21 inclusive illustrate that the anatomic body part penetrated is typically a blood vessel or a cardiac chamber 100. The targeted body part 100 has at least two walls 102, 104 and an internal spatial organ volume 108. This is illustrated in its generic form within FIGS. 16-21.

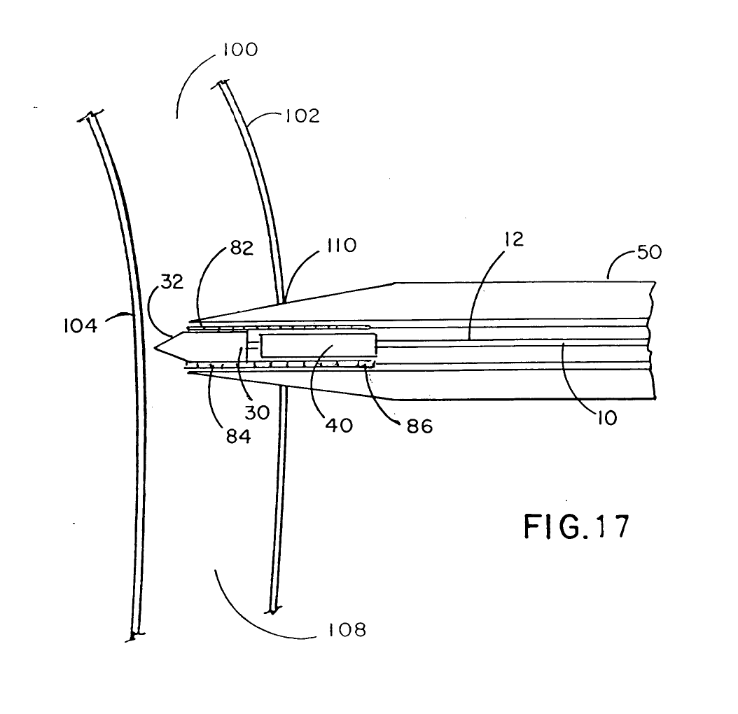

FIG. 16 shows the complete introducer assembly as it approaches the front sidewall 102 of the blood vessel or cardiac chamber. It will be seen therein that the open end 52 of the volumetric sheath is placed adjacent to and in aligned closure with the perforating headpiece 30 of the perforator instrument. The prepared communication channel 80 lies entirely within the internal spatial volume 58 of the volumetric sheath 50 as does the balloon appliance 40 and the supporting shaft 12 of the perforator instrument. Also, as shown by FIG. 17, the balloon appliance is in the deflated state thereby permitting the entirety of the perforator instrument 10 and the penetrating tip 32 in particular to pass out of the enveloped spatial volume provided by the volumetric sheath 50; then to cut into the sidewall 102; and thereby form an aperture 110. The introducer assembly as a whole is then advanced forward through the newly formed aperture 110.

FIG. 17 also shows the position of the prepared inflow or outflow conduit as an integrated unit through the aperture 110 in the front wall 102 of the blood vessel or cardiac chamber. As seen therein, the volumetric sheath 50 housing the linking connector 82 has been pushed forward such that the first cuff portion 84 lies positioned within the internal spatial volume of the blood vessel or cardiac chamber 100; and the perforating headpiece 30 and the deflated balloon appliance 40 have also been extended into the internal spatial volume 108 and thus support the prepared communication or access conduit in this position.

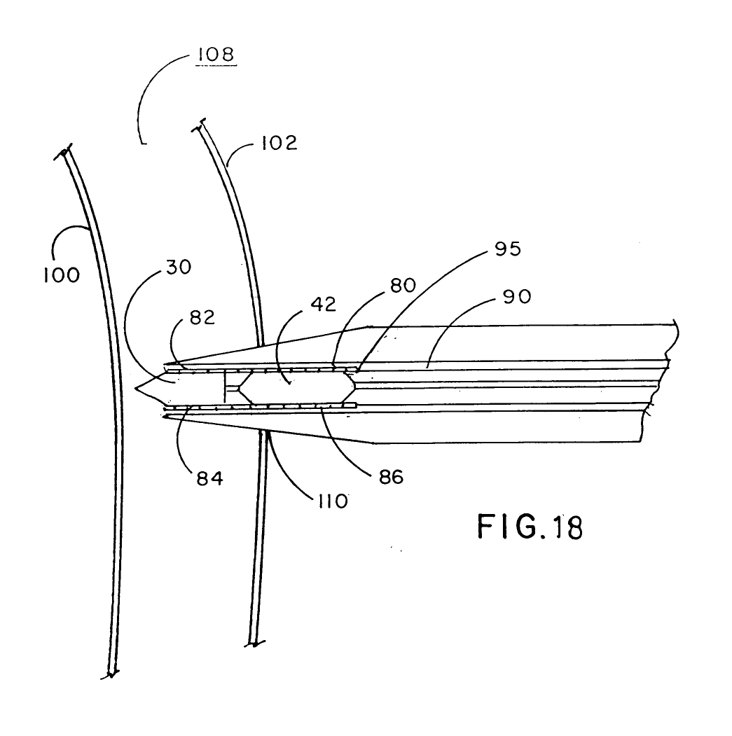

The balloon appliance then is preferably inflated by introducing fluid via the luer lock fitting (not shown) which is passed through the inflation line and inflates the balloon interior space 42 thereby holding the prepared communication or access conduit 80 in place within the aperture 110 itself. This is illustrated by FIG. 18.

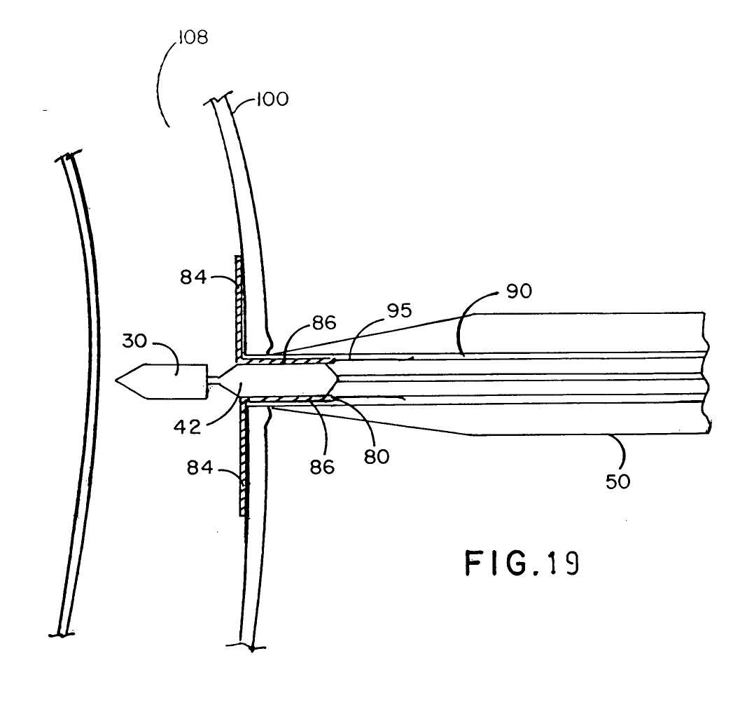

Accordingly, the linking connector 82 which has been permanently joined to the internal sidewall surface 95 of the tubular conduit 90, is then allowed to deform on-demand and deploy in-situ. This event is shown by FIG. 19. The individual acts of deformation and deployment of the first cuff portion 84 within the internal spatial volume 108 of the blood vessel or cardiac chamber 100 thus serve to secure the prepared communication or access conduit 80 to the interior of the anatomic body part; and concurrently places the secured communication channel 80 now accessing and in fluid flow communication with the internal spatial volume 108 of the blood vessel or cardiac chamber. Moreover, while the act of deployment within the internal spatial volume 108 occurs as illustrated by FIG. 19, the tubular conduit permanently joined to the second conduit retaining portion 86 remains in place and in a somewhat expanded state by superelasticity, thermoelasticity, and/or balloon inflation. This retained portion 86 permanently joined to the sidewall of the tubular conduit retains and secures the tubular conduit 90 for unobstructed fluid flow communication.

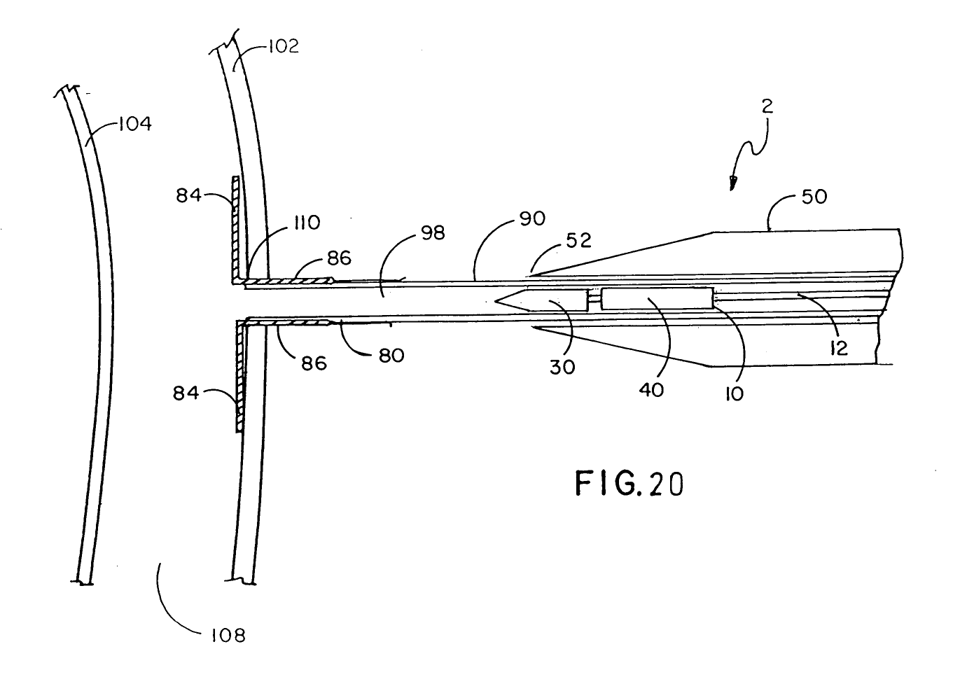

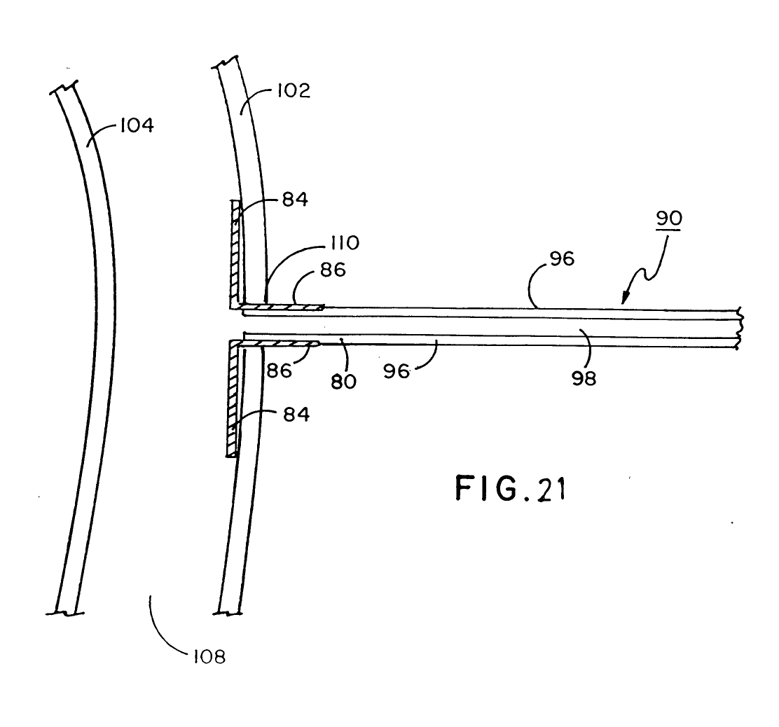

The final stages of the method and system are illustrated by FIGS. 20 and 21 respectively. FIG. 20 shows the introducer assembly being withdrawn after deflation of the balloon from within the internal lumen 98 of the tubular conduit 90. FIG. 21 illustrates the final desired result and shows the sutureless juncture of the prepared communication channel 80 in position through the aperture 110 in the front wall 102 of the blood vessel or cardiac chamber 100.

As seen therein, the prepared inflow or outflow conduit is joined to the interior space of the blood vessel or cardiac chamber; is secured in a fluid-tight manner to the internal spatial volume 108 of the blood vessel or cardiac chamber interior; and is in fluid flow communication with the interior space of this anatomic body part. The linking connector 82 shows the first cuff portion 84 in the deformed state within the interior space of the blood vessel or cardiac chamber and shows that this in-situ deformation acts to secure the tubular conduit 90 to the interior spatial volume of the blood vessel or cardiac chamber and places the prepared communication or access conduit in fluid flow communication for whatever purpose is desired by the surgeon for his patient.

### B. Alternative Embodiments and Formats

The preferred embodiment described previously herein is merely one structural assembly format whose component parts may be alternatively configured for a variety of purposes. To demonstrate the variety of alternative embodiments and structural formats, the following structural designs and constructions are provided. It will be expressly understood, however, that these described alternative embodiments and constructions are merely illustrative of the wide range and broad variety of alternatives which is well within the skill of the ordinary person skilled in this technical field; and that the described formats are merely representative examples of many other constructions which may be used equally well for a particular medical application or specific patient purpose.

### Alternative Embodiment 1

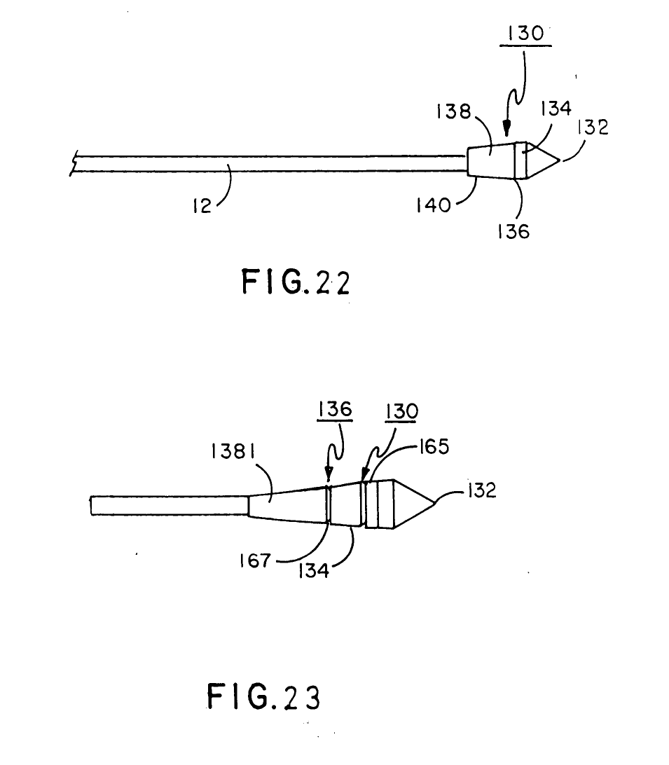

A first alternative design and construction facilitates the passage and removal of the prepared communication or access conduit over the axial length of the perforator instrument and concurrently allows for easy removal of the perforator instrument as well as the introducer assembly as a whole after the communication or access conduit has been joined in-situ to the interior spatial volume of a blood vessel or cardiac chamber. For this purpose a first alternative construction for the perforating headpiece of the perforator instrument is provided as illustrated by FIG. 22.

As seen in FIG. 22, the perforating headpiece 130 now comprises a perforating cutting tip 132, a penetrating body 134 of diminished dimensions and size in comparison to that described previously herein; has a base aspect 136 which is now serving as a surface for a cone-shaped end element 138. As before, the perforating headpiece 130 is integrally joined to the supporting shaft 12 of the perforator instrument. In this construction, the point of juncture and integral union for the perforating headpiece 130 as a unit is at the cone-shaped end element 138.

The benefit and major advantage of this construction is that the cone-shaped base end element 138 is tapered along its sides 140; and that this tapered sidewall 140 for the cone-shaped end element 138 will not only permit easier passage and withdrawal through the linking connector; but also, if necessary, dilate the linking connector structure to permit an unobstructed withdrawal of the perforating headpiece 130 after the communication or access conduit has been joined to the blood vessel or cardiac chamber interior space. If desired, the entire external surface of the perforating headpiece and the sides 140 of the cone-shaped base end element 138 in particular may be covered with a hydrophilic coating in order to provide a more slippery surface and ensure an easier passage.

### Alternative Embodiment 2

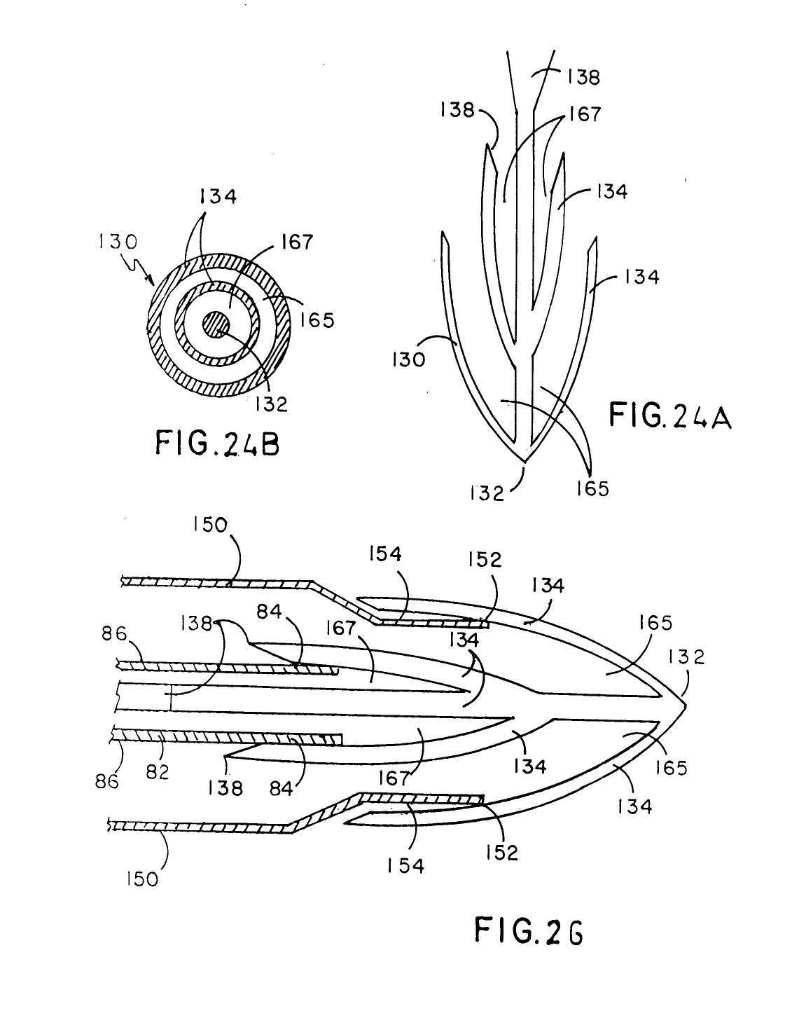

This second alternative embodiment and structural construction is illustrated by FIGS. 23-26 respectively. There are two essential parts to this second alternative embodiment. The first is revealed by FIGS. 23, 24A, and 24B respectively which reproduce in part the perforating headpiece 130 illustrated by FIG. 22 and described herein previously. In this alternative construction, the perforating headpiece 130 again includes a perforating tip 132, a penetrating body 134, a base aspect 136, and a cone-shaped end element 138; but also now comprises a plurality of recesses which individually appear as a groove 165 and a furrow 167 within the penetrating body portion 134 and the base aspect 136 respectively.

Particular details of this structural construction are shown by FIGS. 24A and 24B respectively. As seen therein the recessed groove 165 is circumferentially extensive and deep within the penetrating body 134. Similarly, the recessed furrow 167 circumferentially penetrates sharply through the base aspect 136 and the interior of the penetrating body 134. The cross sectional view illustrated by FIG. 24A shows the manner in which the recessed groove 165 and recessed furrow 167 exist in depth; in comparison, the cross-sectional view of the perforating headpiece 130 (looking forward from the supporting shaft towards the perforating tip 132) of FIG. 24B shows the concentric ring nature and annular alignment of the recessed groove 165 in comparison to the recessed furrow 167.

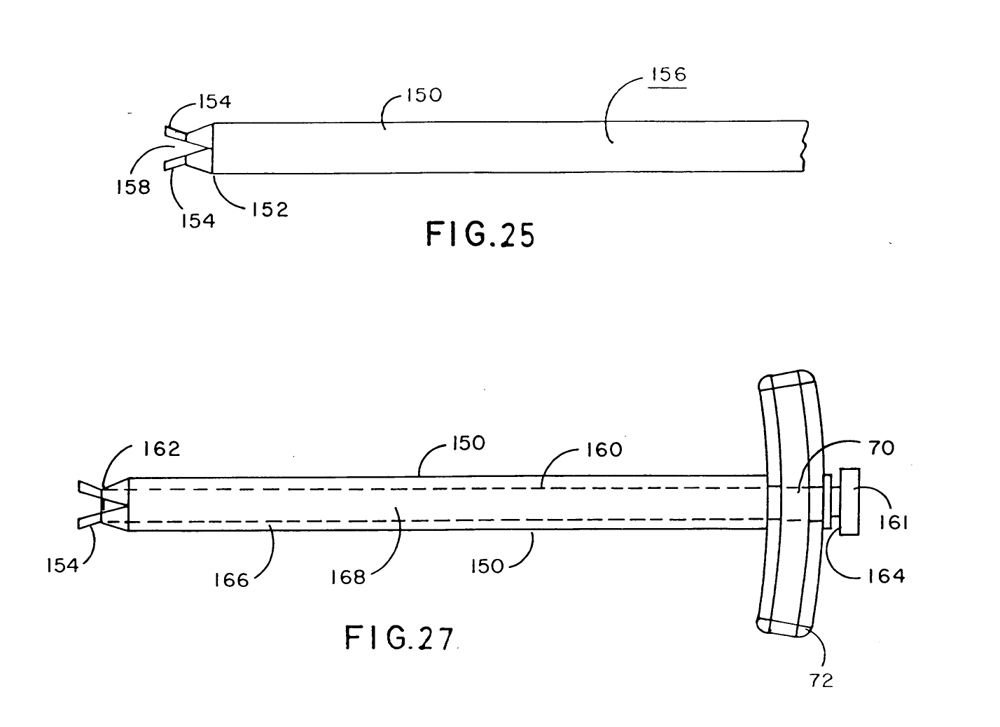

This second alternative embodiment of the perforating headpiece 130 having recessed groove 165 and recessed furrow 167 is intended to be employed with a modified construction for the volumetric sheath illustrated by FIG. 25. In this modified design structure and construction, the volumetric sheath 150 has a front open end 152 which is configured as multiple segmented tangs 154. The multiple segmented tangs 154 are preferably evenly spaced around the circumference of the open end 152 and are desirably biased such that the preferred positioning of the segmented tangs is in the open position as shown in FIG. 25. The multiple biased segmented tangs 154 when compressed annularly into the closed position will form a single circular and unified open end 152; and while in the closed position will provide a unitary opening 152 for the entirety of the volumetric sheath 150 despite being constructed as multiple segmented pieces. In this manner, the segmented tangs 154 will remain preferably in the open, biased position; but at will can be compressed to form a single circular or annular front end opening 152 and access to the interior spatial volume of the volumetric sheath 150.

The positioning of the multiple segmented tangs 154 in the closed position is intended for placement within the recessed groove 165 of the perforating headpiece 130 illustrated previously in FIGS. 23 and 24 respectively. The segmented tangs 154 will fit into and be held by the recessed groove 165; and form itself within the interior space of the groove as the unitary annular opening 152. This is shown by FIG. 26.

In addition, the recessed furrow 167 will receive and hold the first cuff portion 84 of the linking connector 82 after it has been permanently joined to the tubular conduit as the prepared communication or access conduit. The placement of the linking connector 82 at the first cuff portion 84 into the recessed furrow 167 is also illustrated in FIG. 26. This linking connector placement thus allows a further degree of certainty and safety for the prepared communication or access conduit after it has been positioned around the supporting shaft of the perforator instrument and has been enveloped by the volumetric sheath 150.

### Alternative Embodiment 3

A third alternative construction provides a variant format for the volumetric sheath of the introducer assembly. This third alternative construction is illustrated by FIG. 27 and utilizes in part the volumetric sheath structure illustrated by FIG. 25 and described in detail previously herein. In this alternative embodiment, however, the variant structure includes inner sleeve 160 which is of predetermined dimensions and substantially cylindrical configuration. The inner sleeve 160 comprises a open front end 162, an open rear end 164, and a cylindrically-shaped grip 161 joined to the rear end 164. Not only does the inner sleeve 160 slide forward and rearward at will within the interior volume of the volumetric sheath 150; but as the inner sleeve 160 is slid forward towards the segmented tangs 154, the front end 162 engages the segmented tangs 154 of the volumetric sheath 150 and forces the tangs open as a consequence of the physical engagement. This allows quick and easy removal of the volumetric sheath 150 from the introducer assembly, especially after the segmented tangs 154 have been placed in the closed position forming a unitary annular front end.

One major benefit and advantage of this alternative construction using the inner sleeve 160 as illustrated within FIG. 27 is that this format allows the volumetric sheath 150, the outer sheath covering, to be made of a woven synthetic textile material which is prepared in advance and coated with a non-porous polymer coating. The polymer coating would preferably bias the woven textile material of the outer volumetric sheath in the closed position in which the multiple segmented tangs would reform as a single annular opening. Thus, as the inner sleeve is advanced within the outer volumetric sheath, it would effectively expand the polymer coated woven textile material and permit removal of the outer volumetric sheath in a far easier fashion.

Clearly this type of construction and format allows for a volumetric sheath which is composed or designed using a woven synthetic textile material; and thus allows a fabric type construction and a fabric arrangement for the outer sheath which acts as the protective barrier and covering around the perforating instrument. This type of woven textile construction and embodiment for the volumetric sheath, with or without the presence and use of an inner sleeve as shown within FIG. 27, is merely one variant of the many different constructions and materials which may be employed with the introducer assembly as a whole.

### Alternative Embodiment 4

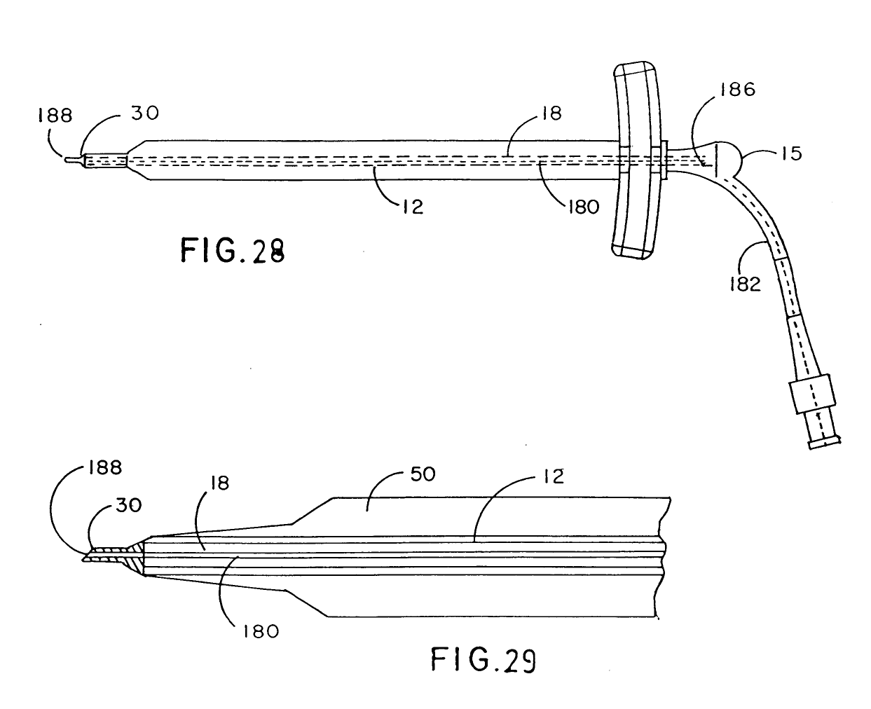

A fourth alternative design and construction is illustrated by FIGS. 28 and 29 respectively. This format and structural design permits the surgeon to utilize the Seldinger technique, a favored procedure for this kind of surgery. In this technique, a guidewire is positioned in the targeted blood vessel or cardiac chamber; and it is this guidewire which is then utilized as the means for precise guidance and placement of the introducer assembly as a whole at that precise anatomic location. For this purpose the alternative construction of FIGS. 28 and 29 is added to the first preferred embodiment previously described herein.

As illustrated, the perforator instrument is comprised of the supporting shaft 12, the perforating headpiece 30 and the knob handle 15. However, within the internal lumen 18 of the support shaft 12, a second hollow lumen 180 exists which extends and passes through the axial length of the perforator instrument 10. This is shown by FIG. 28. The hollow lumen 180 for passage of the guidewire extends through the perforating headpiece 30, through the supporting shaft 12 over its axial length, and exits adjacent to the handle 15 where it is joined to flexible tubing 182. The flexible tube 182 is joined to the hollow lumen 180 at the juncture point 186; and the flexible tube 182 provides an entry portal 184 through which the guidewire exits. A cross sectional view of this internal arrangement, the perforating headpiece end, is illustrated by FIG. 29. The use of the Seldinger technique and the ability to pass a guidewire from the anatomic targeted site at the blood vessel or cardiac chamber directly through the perforating tip of the perforating headpiece and continuously through the entirety of the introducer assembly provides a major advantage and benefit for the assembly.

### V. Details of the Prepared Inflow Conduit and the Prepared Outflow Conduit in Fluid Communication with a Ventricular Assist Device (VAD)

### A. The Linking Connector

An essential component part of the prepared inflow and outflow conduits extending from and in fluid communication with the VAD is the presence and use of a superelastic and/or thermoelastic linking connector, which is preferably comprised of a shape-memory alloy composition.

The shape-memory metal alloy compositions preferably used with the present invention constitute conventionally known blends and formulated metallic mixtures of nickel and titanium which undergo a phase transition—that is, a molecular rearrangement of atoms, molecules or ions within a lattice structure—due to a temperature change. The unique capability of shape-memory alloys is that these alloys are extremely elastic, flexible, and durable; these alloys change shape or configuration as a direct consequence of a change in temperature; and the alloy composition “remembers” its earlier and specifically prepared shape because the phase change affects its structure on the atomic level only, without disturbing the arrangement of the molecules which would otherwise be irreversible.

Superelasticity and Thermoelasticity

When these shape-memory alloys are intentionally superheated far above their transition temperature (either electrically or by external heat), a stretched temperature transformed alloy format results which contracts and exerts considerable force; and the temperature transformed alloy composition will become memory-shaped (deformable in-situ) in a fixed specific configuration. Afterwards, when cooled to below its transition temperature, the prepared alloy composition presents superelasticity properties which allow the alloy to be bent and shaped into other configurations while retaining the fixed “memory” of the particular shape in the earlier superheated condition, the thermoelastic properties. Thus, these shape-memory alloy compositions are recognized as being both superelastic and thermoelastic compositions of matter.

Some preferred alloy formulations

At least twenty different formulations of superelastic and thermoelastic alloys are conventionally known, all of which comprise different mixtures of nickel and titanium in varying percentage ratios [Design News, Jun. 21, 1993 issue, pages 73-76]. These metal alloys are conventionally utilized today in the manufacture of diverse products. For example, a range of different shape-memory alloy wires are commercially available in diameters from 0.001-0.010 inches [Dynalloy, Inc., Irvine, Calif.]. In addition, surgical anchors having such superelastic properties and formed by two or more arcs of wire strands (which can withstand strains exceeding 10%) have been developed [Mitek Surgical Products, Inc., Norwood, Mass.]. Also, blood clot filters formed of superelastic shape-memory alloy wires are commercially sold for implantation in large blood vessels such as the vena cava [Nitinol Medical Technologies, Inc., Boston, Mass.]. While these commercially available products illustrate the use of one or more superelastic and thermoelastic properties as particular articles, a more general listing of conventionally known properties and characteristics for NiTi shape-memory alloy compositions is provided by Table 1 below.

TABLE 1
Conventionally Known Properties of NiTi Shape-Memory Alloys

| | |
|---|---|
| Transformation Properties | |
| Transformation Temperature | −200 to −110° C. |
| Latent Heat Of Transformation | 5.78 cal/g |
| Transformation Strain (for | |
| polycrystaline material) | |
| for a single cycle | 8% maximum |
| for 10² cycles | 6% |
| for 10⁵ cycles | 4% |
| Hysteresis* | 30 to 50° C. |
| Physical Properties | |
| Melting point | 1300° C. (2370° F.) |
| Density | 6.45 g/cm³ (0.0233 lb/in³) |
| Thermal Conductivity | |
| austenite | 0.18 W/cm @ ° C. (10.4 BTU/ft |
| | @ hr @ ° F.) |
| martensite | 0.086 W/cm @ ° C. (5.0 BTU/ft |
| | @ ° F.) |
| Coefficient of Thermal | |
| Expansion | |
| austenite | 11.9 × 10⁻⁶/° C. (6.11 × 10⁻⁶/° F.) |
| martensite | 6.6 × 10⁻⁶/° C. (3.67 × 10⁻⁶/° F.) |
| Specific Heat | 0.20 cal/g @ ° C. (0.20 BTU/lb |
| | @ ° F.) |
| Corrosion Performance** | excellent |
| Electrical Properties | |
| Resistivity (D) | |
| [resistance = D @ length/ | |
| cross-sectional area] | |
| austenite | −100 :S @ cm (−39.3 :S @ in) |
| martensite | −80 :S @ cm (−31.5 :S @ in) |
| Magnetic Permeability | <1.002 |
| Magnetic Susceptibility | 3.0 × 10⁶ emu/g |
| Mechanical Properties | |
| Young's Modulus*** | |
| austenite | −83 GPa (−12 × 10⁶ psi) |
| martensite | −28 to 41 GPa (−4 × 10⁶ to 6 × 10⁶ psi) |
| Yield Strength | |
| austenite | 195 to 690 MPa (28 to 100 ksi) |
| martensite | 70 to 140 MPa (10 to 20 ksi) |
| Ultimate Tensile Strength | |
| fully annealed | 895 MPa (130 ksi) |
| work hardened | 1900 MPa (275 ksi) |
| Poisson's Ratio | 0.33 |
| Elongation at Failure | |
| fully annealed | 25 to 50% |
| work hardened | 5 to 10% |
| Hot Worability | quite good |
| Cold Workability | difficult due to rapid work hardening |
| Machineability | difficult, abrasive techniques are |
| | preferred |

*Values listed are for a full martensite to austenite transition. Hysteresis can be significantly reduced by partial transformation or temary alloys.
**Similar to 300 series stainless steel or titanium.
***Highly nonlinear with temperature

All the different specific formulations and metallic blends comprising nickel and titanium which yield a deformable, thermoelastic, shape-memory alloy composition are suitable for use when practicing the present methodology. All of these shape-memory alloys rely on a crystal phase change from a higher temperature Austenite form to a lower temperature Martensite form to accomplish the memory effect. The cubic Austenite phase behaves much like ordinary metals as it deforms. In contrast, the complex crystal Martensite form can be found by reversible movement of twin boundaries to change the average “tilt” or strain in each segment of the alloy. The overall strain can be eliminated by releasing the stress, by maintaining it if it is not thermally stable (the superelastic effect), or by heating the alloy to change it back to Austenite form (shape-memory effect).The crystal transformation of shape-memory alloy compositions is, by definition, thermoelastic—i.e., it progresses in one direction on cooling below the transition temperature and in the other direction upon heating above the transition temperature. The amount of transformation change versus temperature, measured either as the percent of Martensite form or the strain in a constantly stressed element, is a function of and can be plotted against temperature (° C.) directly; and the change from one phase(and identifiable shape) to another typically occurs in a narrow temperature range (often 5-10° C.). Hysteresis takes place before the reverse transformation occurs.

The amount of strain accommodated due to the movement of twin boundaries, differs in each metallic alloy blending system. In the nickel-titanium system for example, up to 8% reversible tensile strain is available; however, to guarantee a long life use, the strain is often limited to 4-5%.

The stress-strain behavior of shape-memory alloy compositions is employed to help explain the shape-memory effect. For instance, Martensite is much easier to deform than Austenite. Therefore, one can deform the alloy while cold with much less force than when heated to change it back into Austenite form. As a result, the alloy converts thermal energy to mechanical work at high forces.

An essential component part of the apparatus and method for creating a bypass graft is the presence and use of a deformable cuff comprised of a shape-memory alloy composition and prepared in advance to recover its shape in-vivo.

Description of Shape Memory Thermomechanics

The following is a description of shape memory alloys and the methods of preparing and delivering medical devices using shape memory thermomechanical properties.

Properly formulated, mechanically worked, and heat-treated nickel-titanium shape memory alloys undergo a reversible martensitic phase transition. Three essential references on the properties of Nitinol are:

C. M. Jackson, H. J. Wagner, and R. J. Wasilewski, 55-Nitinol The Alloy with a memory: Its Physical Metallurgy, Properties, and Applications, NASA Report, NASA-SP 5110, 1972;

T. W. Duerig et al., Ed., Engineering Aspects of Shape Memory Alloys, Butterworth-Heinemann, London, 1990;

T. W. Duerig and A. R. Pelton, “Ti—Ni Shape Memory Alloys”, in Materials Properties Handbook: Titanium Alloys, Ed. R. Boyer, E. W. Collings, G. Welsch, 1994, ASM Publications.

The shape memory alloys have a symmetric high temperature crystal structure, austenite, and a group of deformation-inducing low temperature variants, martensite. The austenite is a cubic lattice structure and the group of martensites have a monoclinic structure which creates a atomic level deformation of the shape. The change in atomic structure, though both are solids, is a phase change. Stress, in particular shear stress, can assist this phase transformation by straining the austenite in the direction of martensite variants. With stress, martensite variants are selected to accommodate the stress and as a result an apparent plastic deformation can be induced.

The shape recovery occurs with the transformation of variant selected martensite to austenite. This strains recovered can by the transformation can be ˜10% though typically are closer to 7%. This recovery of shape occurs only if the temperature is greater than As, the austenite start temperature, and preferably greater than Af, the austenite finish temperature. With increasing temperature above Af, the transformation from martensite to austenite can occur under increasing levels of shear stress. Shape memory alloys use this transformation to recover the austenite shape from the strain accommodating martensite.

Shape memory is the general term for this property, but when the austenite is transformed to the martensite by stress at a temperature greater than Af, the alloy is said to exhibit superelasticity.

These and other related terms, as used in medical devices, have been are compiled in the ASTM Standard F 2005-00, “Standard Terminology for Nickel Titanium Shape Memory Alloys:

Shape memory alloy, n—a metal which, after an apparent plastic deformation in the martensitic phase, undergoes a thermoelastic change in crystal structure when heated through its transformation temperature range resulting in a recovery of the deformation.

Superelasticity, n—nonlinear recoverable deformation behavior of the Ni—Ti shape memory alloys at temperatures above the austenite finish temperature.

Discussion—The nonlinear deformation arises from the stress-induced formation of martensite on loading and the spontaneous reversing of this crystal structure to austenite upon unloading.”

Shape memory occurs when variant selected martensite at T<As transforms to austenite at T>Af. The variant selection is seen as apparent plastic deformation. The advantage of shape memory is apparent during the process of loading a device into a catheter—the deformed martensite is stable and more easily put into the catheter. Examples include devices with an Af=25 C placed in a catheter at say 20 C or Af=0 C placed in a catheter at say −10 C.

Medical devices delivered by a catheter may use shape memory deployment by the following procedure: Form martensite thermally by cooling below Mf, the martensite finish temperature, say by immersing in liquid nitrogen. The cooling forms the martensite. Then, while maintaining the temperature below As, deform the device and place into the delivery system. The deformation of the martensite results in apparently plastic deformation, by martensite variant selection, to accommodate the large strains imposed. The device can then be delivered at the desired location in the body. An isotonic saline solution or other cooling means may be used to reduce the forces on the device to ease delivery through and out the catheter.

Superelasticity occurs when variant selected martensite at T>Af transforms at T>Af. The advantages of a superelastic device are that a low Af material may be loaded into the catheter at T>Af and the low Af material has a greater force at body temperature. For example, a NiTi device with an Af=0 C may be loaded into a catheter at 21 C. Low Af materials have greater driving stresses on deployment in vivo. For example, a device with an Af=0 C may have a recovery stress of 40,000 psi at 37 C whereas a device with an Af=25 C the recovery stress may be only 10,000 psi at 37 C. The disadvantage to a low Af device is the increased difficulty in placing the device in the catheter at temperatures at temperatures above Af. The difficulty is the result of the unstable deformation-induced martensite which has to be restrained with force to prevent it from springing back to the austenite shape.

Medical devices delivered by a catheter may use superelasticity by the following procedure: At a temperature greater than Af deform the device into the delivery system. This will result in the formation of martensite variants, stable only under sufficient stress in the proper direction, to accommodate the large strains imposed. Then deliver the device at the desired location in the body. An isotonic saline solution or other cooling means may be used to reduce the forces on the device which may make the delivery easier.

The broad case of variant selected martensite formation at T<Af reversing to austenite at T>As occupies a middle ground between shape memory and superelasticity. The advantage of this method of device delivery is that it avoids the step of cooling the device to Mf but yet the device will retain some shape changing variant selected martensite after deforming into the delivery shape making the loading easier.

Medical devices delivered by a catheter may use this shape setting by the following procedure: From unstressed state and a temperature greater than Af, cool the device to a temperature between Mf and Af. Then deform the device into the delivery system. This will result in the transformation of austenite to thermally stable martensite variants to accommodate the large strains imposed. Then deliver the device at the desired location in the body. An isotonic saline solution or other cooling means may be used to reduce the forces on the device which may make the delivery easier.

This method of using Nitinol was illustrated by Andrew Cragg, et al., in “Nonsurgical Placement of Arterial Endoprosthesis: A New Technique Using Nitinol Wire”, Radiology 147:261-263, Apr. 1983. The article states that the wire “ . . . transformed over a broad temperature range (25 to 38 C) . . . ” If we assume this is the range of Af for the various devices tried and we use the typical range of differences in Af and Mf, Af−Mf={40, 60}C, then the resulting range of Af's of his devices lie in the range of Mf in {2, −35C}. Thus though possible, it both unlikely and not critical, that the device was transformed to martensite before stressing. By this publication, Cragg taught the use of stress-induced martensite to load and deliver catheter based medical devices.

At body temperature, the mechanically constrained device upon deployment from the catheter will recover part of its shape via the transformation from martensite to austenite. Thus As will be below body temperature, 37+/−1 C. Depending on the material dimensions and the stress needed, the device's Af will be in the range of −10 to 10 C for high stresses, 10 to 25 C for moderate stresses, and 25 to 35 C for low stresses.

It is recognized that plastic deformation increases with increasing strain and with increasing temperature above Af, thus the high stress devices may exhibit poorer shape recovery from the deployment device.

This plastic deformation at high temperatures excludes the variety of binary NiTi alloys prepared in such a way that the device's Af is much less than 37C, say Af<−30 C. With such a low transformation temperature, when the device is the catheter at body temperature large strains result in true plastic deformation of the austenite and not martensite accommodation. This is a result of the increase in stress with temperature at which the austenite to martensite occurs. At a temperature approximately 50 to 60 C above Af, the stress at which austenite is plastically deformed is approximately equal to the stress of austenite to martensite transformation. At approximately 60 to 100 C above Af, mechanically constrained martensite will either revert to austenite and be plastically deformed, or will be at such high stress that there will be plastic deformation of the martensite.

The devices austenite shape is typically set by heating the device in its desired shape to temperatures between 300 and 600 C, preferably 460 to 520 C. This heating is typically done for a short period of time to maintain some of the forming processes induced strength. Typical exposure time are 1 minute to 1 hour, preferably 1 to 5 minutes, depending on the mass of the fixture holding the deformed device.

To minimize the amount of leachable metals in-vivo, the NiTi surface is preferably polished after heat setting. Following polishing, thorough rinsing followed by a passivating treatment with nitric acid to remove leachable Ni ions is typically done. ASTM Standard A-967-96, Standard Specification for Chemical Passivation Treatments For Stainless Steel Parts provides a variety of treatments that have been found to be a good starting point for passivating NiTi alloys as well.

The devices described in the patent may use either or both shape memory and superelasticity behaviors in preparation, delivery, and use. Since shape memory is the inclusive term, it is used exclusively to describe the devices.

The Range and Variety of Useful Alloy Formulations

Many formulations of NiTi, NiTiX tertiary and NiTiXY quaternary alloys are prepared by the primary melters such as Special Metal Corporation, New Hartford, N.Y.; Wah Chang, Albany Oreg.; and Furukawa Electric, Japan. They supply bar stock or other wrought products to secondary processors such as Shape Memory Applications, Inc., Santa Clara Calif.; Fort Wayne Metals, Fort Wayne, Ind.; or Memory Corporation, Brookfield Conn. The primary melters and the secondary suppliers provide a variety of wire, sheet, and tube products to medical companies for using in making devices. Currently available devices include surgical anchors [Mitek Surgical Products, Inc., Norwood, Mass.]; blood clot filters for implantation in large blood vessels such as the vena cava [Nitinol Medical Technologies, Inc., Boston, Mass.]; and stents [Nitinol Devices Incorporated, Fremont Calif].

Ni—Ti based alloys with ternary and quaternary additions of V, Cr, Fe, Cr, Co, Cu, Zr, Nb, Mo, Pd, Hf, Ta, and Pt have all been reported in the literature. See, for example, the proceedings of the Shape Memory and Superelastic Technologies International Conferences, SMST, 1994, 1997, and 2000, and the proceedings of the International Conferences on Martensitic Transformations, ICOMAT 1989, 1992, 1995, and 1999. These alloys have allowed both raising and lowering the transformation temperature while maintaining ductility, both increasing or decreasing the stress and temperature hysteresis in the alloy, reduced or enhanced the appearance of a third (intermediate) phase, or improved corrosion characteristics.

Other, not NiTi based, shape memory alloys are known and new ones are being developed. The above conference proceedings provide a good picture of the state of development of these alloys. Most of these alternative alloys typically have neither the great corrosion resistance nor the large strain capability of NiTi alloys. The iron based alloys, if commercialized, would provide a less expensive material for making the devices. The beta titanium alloy, beta Ti Mo Al Cr V Nb disclosed in U.S. Pat. No. 6,258,182, possess a recovery up to 3.5% strain, a low stiffness and greater formability than NiTi. Despite these compromises in strain recovery and strength, this alloy may be useful for patients with sensitivity to the nickel in NiTi.

For increasing the strength or lowering the volume of the cuff connector, it may be useful to combine NiTi with stiffer materials such as titanium alloys, CoCrMo alloys, or stainless steels. These materials may be combined with NiTi by welding, soldering, mechanical joining or use of epoxies and adhesives.

Fixing the Memory-Shaped Configuration in the Metal Alloy

To prepare and fix the particular (or desired) shape to be “remembered” when the alloy undergoes a temperature phase transition, the alloy composition must be superheated initially to about 500° C. (or roughly 930° F.) for an hour while held in the fixed shape and position to be memorized. During the superheating process, the native alloy blend enters what is called the Austenite phase—a rigid lattice of nickel atoms surrounded by titanium alloys. Then, as the alloy metal cools below its transition temperature (which will vary with the percentage proportions of nickel and titanium), the alloy composition adopts the Martensite phase, in which the nickel and titanium atoms assume a very different arrangement—one that is very easy to bend and deform. Subsequently, when the deformed metallic alloy is reheated to the chosen transition temperature range between

25-35° C., thermal motion causes the atoms to snap back into the Austenite phase, thereby restoring the fixed memory-shaped configuration of the object [Invention & Technology, Fall 1993, pages 18-23].

For purposes of practicing the present invention, it is most desirable that the shape-memory alloy composition be prepared in a metallic blend and formulation such that the temperature transition phase occurs at a temperature less than about 35° C.; but greater than about 25° C.; and preferably be in the range from about 30-35° C. This preferred 30-35° C. transition phase temperature range is dictated by the demands of the human body which maintains a normal temperature at about 37° C. (98.6° F.); and typically shows a normal temperature range and variance of one or two degrees Celsius above and/or below this normative temperature standard. It is for this reason that the broad temperature range be about 25-35° C. and the preferred temperature transition occur in the range of 30-35° C.; but that such transformation into the intended and fixed memory-shaped configuration occur at least by a temperature of 35° C. to insure a safety margin of medical usefulness.

### B. Thermoelastic Properties of the Linking Connector

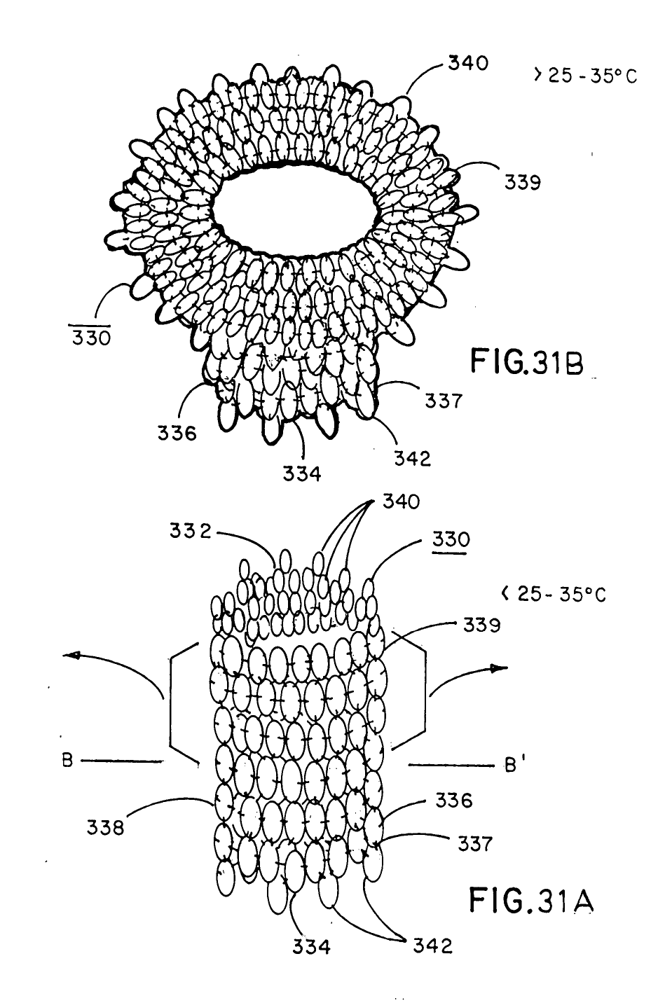

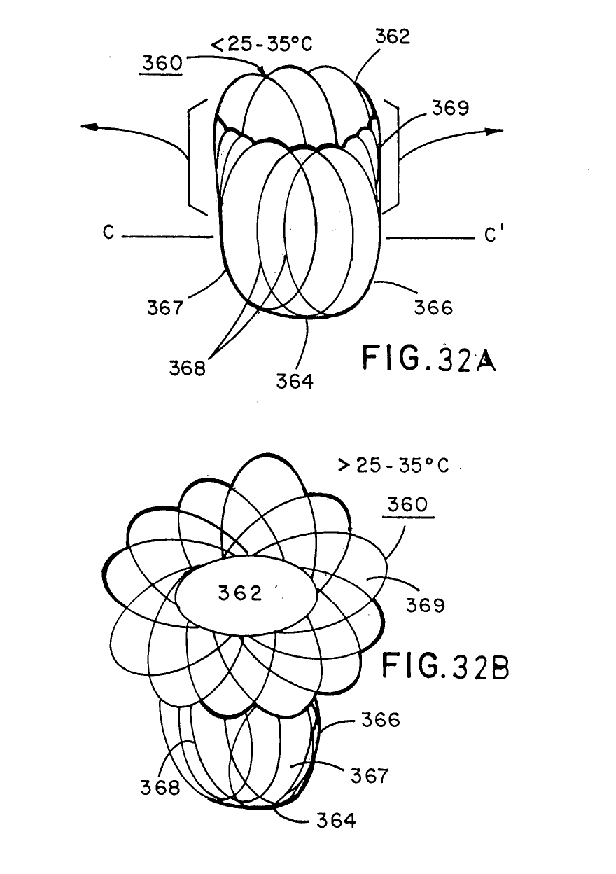

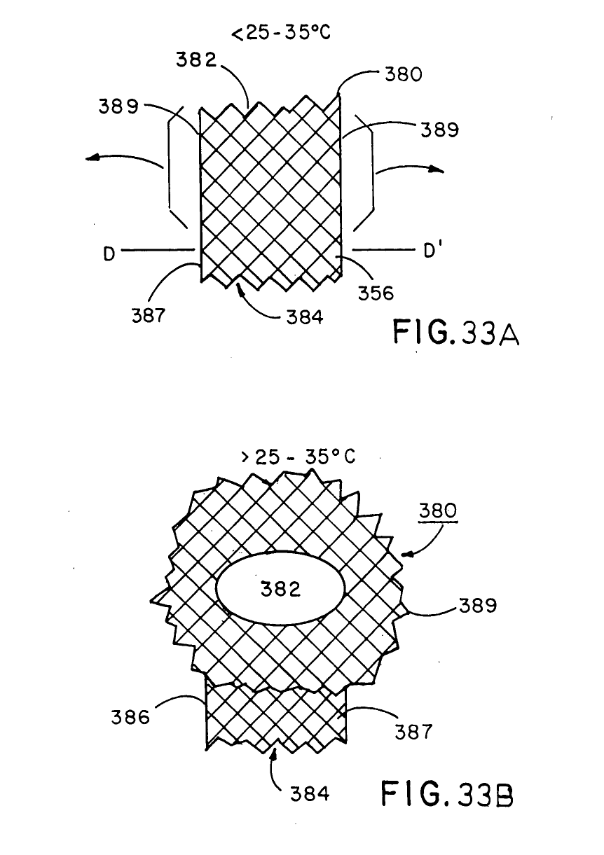

The shaped connector configurations of the thermoelastic alloy composition at temperatures less than about 25-35° C. (a temperature below its transition temperature at which the alloy exists in the Martensite phase) may take a broad variety of different lengths, diverse dimensions, and disparate overall configuration. Merely exemplifying the range and diversity of three-dimensional forms into which the alloy compositions can be shaped into a linking connector structure at temperatures below 25° C. are those illustrated by FIGS. 30-33 respectively. For purposes of practicing the present invention, FIGS. 30-31 are considered more preferred embodiments and constructions of the shaped alloy structures, while FIGS. 32-33 respectively represent formats and fabrications of the deformed in-situ alloy compositions in less frequently utilized shaped configurations.

Effect of Temperatures Less than and Greater than 25-35° C.

As illustrated and embodied by FIGS. 30A and 30B, the deformable in-situ, thermoelastic linking connector is a substantially cylindrical-shaped collar which is open at each of its ends 302, 304. The linking connector 300 is hollow; is substantially round or oval (in cross-sectional view); and has a determinable first configuration and dimensions initially which are deformed at will into a second memory-shaped configuration when placed at a temperature greater than about 25-35° C.

It is most desirable that the thermoelastic material constituting the sidewall 306 of the connector 300 be prepared and shaped as a first-configuration along the axis AA′ as shown within FIG. 30A; and that the thermoelastic material constituting the sidewall 306 be an open-weave pattern of a memory-shaped alloy rather than take form as a solid mass of thermoelastic alloyed material. For this reason, the sidewall 306 illustrated within FIG. 30A appears in the first configuration as an open meshwork of wires 308 which are intertwined to form a substantially hexagonal pattern. This open meshwork of wires 308 provides the desired resiliency, flexibility, and memory-shaped deformation capability (particularly along the axis AA′) such that the first or upper cuff portion of the sidewall 306 will become deformed and flared outwardly on-demand to yield the memory-shaped second configuration constituting the flared-lip deformity 310 shown by FIG. 30B.

It will be recognized and appreciated that the deformed cuff portion shown by FIG. 30B is merely the result of removing the cuff structure from a temperature less than 25-35° C. and placing it into a temperature environment greater than about 35-35° C. Thus, solely as a consequence of the change in temperature, the uppermost cuff portion 309 of the open meshwork of wires 308 above the axis AA′ has become deformed in-situ such that the upper sidewall 309 adjacent to the open end 302 has expanded outwardly, flared, and become bent into a curved lip configuration in the memory-shaped deformed state.

Note that FIG. 30B shows the upper deformation in the fully deployed state; while the open meshwork of wires constituting the lower retaining portion 307 of the sidewall 306 at the other open end 304 remains relatively stable and substantially unaltered in its original shape and state. Alternatively, however, the lower retaining sidewall portion 307 can be made to expand or diminish slightly so that it will annularly fit more tightly outside of or within the conduit wall. The deformation in-situ thus is controlled thermally and the forces at the upper curve sidewall portion from the AA′ axis cause the outwardly extending, flared lip result as the fully deployed state. Moreover, the resulting flared lip zone 310 retains structural strength and resiliency as an open meshwork of superelastic wires despite having been deformed in-situ and deployed in full. The ability of the first cuff portion to be deformed and deployed in the manner illustrated by FIGS. 30A and 30B respectively is an attribute and characteristic of each embodiment and construction for the thermoelastic linking connector.

The construction and design for the linking connector is an example of the engineering principle that structural form at will follow intended function. As a component part of the system apparatus and methodology for attaching a tubular conduit in-vivo, the functions of the linking connector are twofold in nature: (1) the temperature-deformable linking connector is intended to engage and become joined to either a synthetic duct prosthesis or a previously excised vascular segment which will serve as the tubular conduit in-vivo; and (2) the temperature-deformable linking connector is intended to be positioned within the internal lumen of a blood vessel or within a cardiac chamber cavity such that a portion of the connector wall becomes positioned and secured within the internal lumen (the blood flow channel) of the blood vessel or the interior of the cardiac chamber permanently in a fluid-tight manner. Thus, as illustrated by the embodiments of FIGS. 30A and 30B, the uppermost cuff region 309 of the alloy comprising the linking connector will be deformed on-demand merely by warming the article to a temperature greater than 25-35° C.; and such deformation when deployed into a flared outwardly bent form will become secured within the lumen of the artery or vein or the cavity of the cardiac chamber. Concomitantly, the retained portion 307 will remain permanently joined in substantially unaltered form to the tubular conduit.

Several attributes and characteristics are commonly to be shared among all embodiments and constructions of the thermally deformable and deployable on-demand linking connector. These include the following:

(a) Only a portion of the alloy material constituting the memory-shaped linking connector need be thermally deformable and deployable on-demand. For convenience and greater facility in achieving such temperature initiated deformation in the degree and at the time desired, it is preferred that the alloy composition forming the linking connector be an open weave or wire meshwork rather than a solid sheet alloy mass, which is considered to be more difficult to deform in a thermally-controlled manner. There is, however, no substantive restriction or limitation as such at any time or under any intended use circumstances which necessitates an avoidance of a solid sheet of material, either as a single alloy sheet or as a laminated plank of alloy material. Accordingly, the choice of whether to use an open wire meshwork or a solid sheet of alloy material is left to the discretion of the user.

(b) The thermoelastic linking connector need only be comprised of superelastic, resilient and flexible metallic alloy matter. A number of different alloys of varying formulations may be usefully employed when making a deformable memory-shaped linking connector suitable for use with the present invention. Among the desirable alloy formulations are those characterized by Table 1 above.

(c) After the deformable in-situ and deployable at will linking connector has been manufactured using shape-memory alloy materials, the first configured cuff portion structure (prior to thermal deformation) may be covered to advantage with one or more biocompatible coatings. These biocompatible coatings are intended to water tighten the article and to facilitate the sewing of the tubular conduit to the linking connector as well as to reduce the interactions of the immune system and tissue reaction with the prepared communicating channel after it has been secured in their appropriate locations in-vivo. Such biocompatible coatings are conventionally known; will reduce the severity and duration of immune or tissue reactions which frequently disrupt or interfere with grafts; and are considered desirable in a majority of use instances in order to minimize the body reaction to surgery. A representative listing of biocompatible coatings deemed suitable for use with the deformable thermoelastic connector or communication or access conduits is provided by Table 2 below.

TABLE 2
Biocompatible Coatings

High temperature pyrongen-free carbon;
Polytetrafluoroethylene (PTFE) and other polyhalogenated carbons;
Fibronection;
Collagen;
Hydroxyethyl methacrylates (HEMA);
Serum albumins;
Supraflim (Genzyme Corp.);
Silicone polymer;
Polyurethanes;
Tetrathane (Dupont);
Polytetramethylene polymers;
Dacron;
Polyester woven fabric;
Polycarbonated urethanes;
Heparin;
Antiplatelet agents;
Metal coating;
Anticancer agent;
Antitissue-cell growth factor;
Hormone;
Tissue/cell growth factor;
Antibactorial or antiviral or antifungal agents;
Computer chips for measurement or evaluation of condition
or environment of the human or other biological bodies;
Spider silk proteins;
Fluorocarbon;
Polyethylene oxide;
Sulfonate;
Hydrocarbon;
Polyurethaneurea;
Polylactides;
Polydienes;
Polyolefins;
Rubber;
Sulfonate;
Polyetherurethane;
Thermoplastic silicone;
Shape-memory thermoplastics; and
Mixtures of any or all of the above materials.

(d) Although the configuration of the memory-shaped linking connector prior to thermal deformation (as exemplified by FIG. 30A) may appear as a geometrically regular and coherent structure, there is no requirement or demand that either the detailed structure or overall appearance of any configured connector conform to these parameters. Accordingly, it will be recognized and understood that the deformable and deployable shape-memory alloy structure need not take form as a completely encircling band or collar of thermoelastic material. To the contrary, L-shaped, T-shaped or H-shaped constructions of alloy material where the annular sidewalls do not overlap or join completely and/or where a gapped distance separates the arms of the linking connector are both permitted and envisioned. Moreover, although the isotropic cylindrical-shaped format of the connector illustrated by FIG. 30 is highly desirable in many instances, there is no requirement that the diameter of the connector structure prior to or after thermal deformation be constant or consistent over its entire axial length. Thus, anisotropic structures as well as isotropic constructions are intended and desirable. In this manner, the linking connector in its initial state prior to thermal deformation may have a variable internal diameter over the axial length of the article in which one open end may be either greater or lesser in size than the other open end; and there may be multiple increases and decreases in diameter size successively over the entire axial length of the connector itself. All of these variations in construction and structure are within the scope of the present invention.

To illustrate some of the more common variations and differences available and envisioned for a deformable in-situ and deployable at will linking connector intended for use with the present invention, the alternative embodiments illustrated by FIGS. 31-33 are provided. As shown within FIGS. 31A and 31B, the initial shaped configuration for the thermoelastic structure 330 appears as a cylindrical-shaped article or cuff having two open ends 332, 334 and a rounded sidewall 336. The body of the sidewall 336 is an open meshwork of closed wire loops 338, each closed wire loop being joined at multiple points along its perimeter to at least one other closed wire loop—thereby forming an open grid meshwork.

A notable feature of the connector construction within FIG. 31A is the outer edges of the open ends 332, 334, each of which is formed by a closed wire loop which is more easily bent and thermally deformed in-situ than the closed-loop meshwork in the middle of the sidewall 336. In many instances, the availability of closed-loop edges 340, 342 provide an enormous benefit and advantage when thermal deformation of the linking connector occurs in-situ. In addition, a portion of the article shown by FIG. 31A has been memory-shaped to deform substantially at the midline along the axis BB′ such that the upper sidewall upper portion 339 near the open end 332 and the edge 340 will deform in-situ and flair outwardly as a consequence of placing the sidewall in a temperature environment greater than about 25-35° C.

The result of thermal deformation in-situ at a temperature greater than about 25-35° C. and deployment of the deformation in full is shown by FIG. 31B. The sidewall upper portion 339 has become deformed and bent from the open end 332 to about the midline axis BB′. However, the lower sidewall retainer portion 337 has remained substantially unaltered overall its surface area from the midline axis BB′ to the other open end 334. The full deployment of this memory-shaped second configuration is illustrated by FIG. 31B and represents the thermally deformed structure which attaches and secures a tubular conduit to the internal lumen of an artery or vein in-vivo or into the internal cavity of a cardiac chamber.

A third embodiment of a thermally deformable linking connector is illustrated by FIGS. 32A and 32B. As shown therein, the initial configuration for the deformable linking connector 360 is illustrated by FIG. 32A and appears primarily as a series of coiled wires 368 whose overlapping and intersecting junctures have been fused together to make a coiled unitary article. The deformable article has two open ends 362, 364 and an open coiled sidewall 336 formed from the commonly fused coils of wire. The open lattice work of coiled wires 368 provides the flexible and resilient meshwork suitable for achieving the primary functions of the memory-shaped linking connector. The sidewall 366 also has been pre-stressed along the middle axis CC′ such that the uppermost sidewall portion 369 will become bent and deformed outwardly when exposed to an environment temperature greater than about 25-35° C.

The consequence of placing the coiled linking connector in an ambient temperature greater than about 25-35° C. is shown by FIG. 32B. It will be appreciated that the memory-shaped configuration of FIG. 32B is intended to be an in-situ generated event and result, which can be deployed fully and completely at will. Thus, when fully deformed and deployed, the flared out upper sidewall portion 369 has become bent at nearly a 90 degree angle with respect to the lower retained sidewall portion 367; and the midline CC′ will generally serve as the axis of thermal deformation and deployed curvature for the coiled linking connector.

A fourth alternative embodiment is provided by FIGS. 33A and 33B in which a thermally deformable cuff or band-shaped linking connector 380 is shown having two open ends 382 and 384. In this instance, however, the sidewall 386 of the linking connector is comprised of a solid sheet of alloy material. Two other features are also included in this embodiment of the thermally deformable structure due to its construction using a solid sheet of resilient material as the sidewall 386 for the linking connector. The sidewall 386 has been preferably pre-scored to form cross-hatched squares over the axial length of the sidewall; and the pre-scored sidewall thus will deform far more easily and bend outwardly along the scored lines of demarcation as shown when the linking connector is placed in an ambient temperature greater than 25-35° C. Similarly, the sidewall material has been pre-stressed along the midline axis DD′ such that the upper most region 389 nearest the opening 382 will become bent far more easily and deform in a controlled fashion when and as required by the user.

The effect and consequences of placing the linking connector 380 in an ambient environment whose temperature is greater than about 25-35° C. is shown by FIG. 33B. The uppermost sidewall portion 389 has thermally deformed into the memory-shaped second configuration; and in the fully deployed state has become bent into a curved lip extending outwardly from the midline axis DD′. However, the lower sidewall portion 387 has remained substantially unchanged from its initial shape and size. The memory-shaped deformation characteristics have thus generated an in-situ deformation and deployed configuration most suitable for the attachment and securing of a tubular conduit in-vivo.

### C. Superelastic Properties of the Linking Connector

It will be noted and appreciated also that the superelastic properties and use characteristics of the linking connector as a structural entity exist in addition to and concurrently with its thermoelastic properties as well as the ability to thermoelastically deform in-situ on-demand. The superelastic properties of each linking connector in any of its many structural formats typically include: (a) extreme elasticity in being able to return to its original size and shape after having been stretched, compressed or altered in configuration; (b) resilience in which the strain or energy created by a bending movement, force, torque or shear force and applied to an elastic material is converted and does not cause fragmentation, or cracking, or a mechanical breakdown of the material; and (c) malleability in being able to be mechanically altered in shape or configuration (whether by rolling, forging, extrusion, etc.) without rupture and without pronounced increase in resistance to deformation. For purposes of practicing the present invention, all of the conventional nickel-titanium metallic formulations which are shape-memory alloys as described herein and characterized by Table 1 previously also are alloys which have and present superelastic properties.

The value of employing linking connectors which exhibit superelastic properties, in addition to their demonstrable thermoelastic capabilities, lies in the user's ability to control separately and individually the physical deployment of the linking connector in its intended memory-shaped configuration—in terms of choosing the precise timing, physical location, and exact placement—after thermoelastic deformation and shape-memory reconfiguration of the linking connector structure itself has been initiated. Thus, the act of and means for controlled deployment, the spreading or arranging in appropriate position, for the linking connector is separate and distinct from the thermal initiation and event of thermoelastic deformation on-demand for the linking connector in-situ. The differences are easily illustrated by easy reference to the introducer assemblies (shown by FIG. 15B) and to the method of introducing a prepared communication channel or tubular conduit to a blood vessel or cardiac chamber (as illustrated by FIGS. 16-21 respectively).

It is the user's choice and option, whether by personal intent or necessity, when to allow the linking connector (then joined to the tubular conduit) to reach the critical temperature required for thermal deformation to occur. However, once this critical temperature is reached, thermal deformation and thermally caused alteration of the linking connector transient structure into its permanent memory-shaped configuration will occur—if and only if there is then sufficient physical space and ambient environment room for the act of structural deformation to be performed fully and completely. Yet, if the linking connector (and the joined tubular conduit) lie within a constrained and limited space and/or a close boundary environment at the moment of thermoelastic initiation, then the thermally initiated act of deformation and reconfiguration becomes restrained, incomplete, repressed, and unfulfilled. No physical deployment and actual structural alteration into the shape-memory configuration can or will occur unless and until the physical constraint(s) are removed and the linking connector is released and has sufficient spatial freedom of movement and rotation to complete the act of shape deformation in full and to present the intended shape-memory configuration in an unconfined form.

Accordingly, if for example the critical temperature were reached for the linking connector, the initiation and event of thermoelastic deformation will have occurred and begun in-situ while the linking connector was spatially confined within the internal volume of the sheath; and the first sidewall portion of the linking connector would be physically constrained and be prevented from deforming in full by the limited space and physical obstruction created by the interim diameter size of the volumetric sheath.

It is essential therefore to recognize and appreciate that while thermoelastic deformation in-situ for the linking connector occurred on-demand—that is, within the volumetric sheath of the introducer assembly, the act of physically deploying the thermoelastically activated linking connector was purposefully delayed and the act of thermal deformation itself was restrained and controlled spatially until the moment the user chose for most effective anatomic placement and appropriate local positioning for the memory-shaped configuration. Clearly, it is the superelastic properties of the alloy formulations which provide the user with the capability not only to separate the individual act of thermoelastic deformation in-situ on-demand from the act of spatial deployment and constrained control at will of the spatial deployment of the thermoelastically deforming linking connector; but also to allow the user to choose for himself the precise timing, physical location, and proper placement for the deployment of a thermoelastically deforming linking connector as a direct consequence and result of being able to control such spatial deployment.

### D. The Tubular Conduit

The tubular conduit comprises any biocompatible tube, sleeve, channel, flow line, hose, piping, duct, or configured outlet which allows and provides an unobstructed conveyance and transport of fluid matter or access for other instruments through its interior space.

By definition, the term “fluid matter” includes and encompasses any and all flowing solids, liquids, and/or gases as well as any mixture of these materials without regard to their chemical composition, degree of purity, amassed volume or quantity, and/or medical significance or value. The desired characteristics of synthetic conduits used as communication or access conduits are nonimmunogenicity, easy availability and storage, less risk of kinking (due to its stiffness), a less turbulent flow (due to uniform diameter), and an absence of branches.

The medical value of synthetic conduits in-vivo has been substantially investigated.

The choices of materials recognized as being suitable for the making of a biocompatible synthetic conduit are quite limited. These are provided by Table 3 below.

TABLE 3
Synthetic Conduit Materials
Synthetic Substances

Dacron (knitted or woven) polymer;
Polytetrafluoroethylene or “PTFE” (knitted or woven);
Impra;
Teflon polymer;
Polyvinyl alcohol;
Nylon;
Fluoroploymer fiber;
Keratin protein;
Graphite;
Kevlar polymer;
Polycarbonated urethane;
Sulfonate;
Fluorocarbon;
Hydrocarbon;
Polyethylene oxide;
Polysulfones;
Polylactides;
Polydienes;
Polyolefins;
Polyether;
Polyurethane;
Polyetherurethane;
Thermoplastic silicone;
Shape-memory thermoplastics;
Synthetic or natural rubber;
Silicone;
Thermoplastic polymers and elastomers;
Collagen, human or bovine;
Spider silk proteins;
Mixture of any or all of the above materials.

The tubular conduit has at least one tubular wall of fixed axial length; has at least one proximal end for entry; has at least one distal end for egress; and has at least one internal lumen of a volume sufficient to allow for on-demand passage therethrough of any fluid matter.

Many different types and constructions of tubular conduits are conventionally known and used; and a wide range and variety of tubular conduits are available which are extremely diverse in shape, design, and specific features. All of the essential requirements of a tubular conduit exist as conventional knowledge and information; and all of the information regarding conduit design and described in summary form hereinafter is publicly known, widely disseminated, and has been published in a variety of texts. One or more valves can be incorporated in the tubular conduit. The reader is therefore presumed to be both familiar with and have an in-depth knowledge and a general understanding of conventional tubular conduits.

A number of specific types of tubular conduits are known today; but for purposes of practicing the present invention, a number of newly designed or specifically designed conduits of varying lengths and sizes suitable for use are expected and intended to be developed and manufactured subsequently. Equally important, minor modifications of the presently existing general categories of tubular conduits are equally appropriate and are expected to be found suitable for use when practicing the present invention.

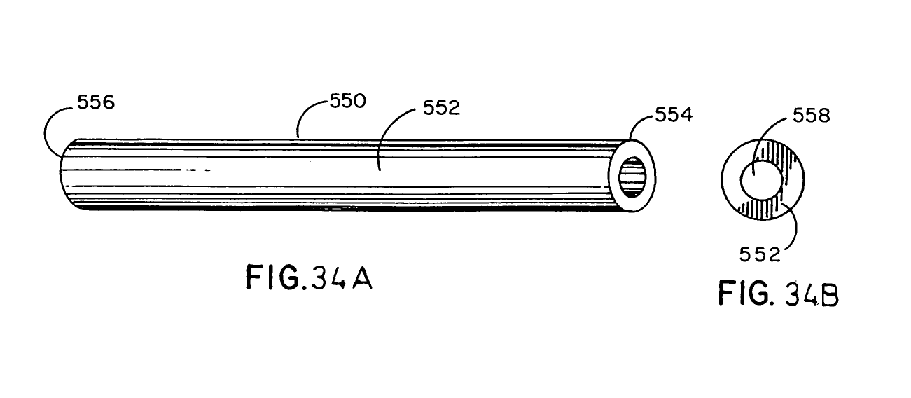

Merely representative of tubular conduits in general without regard to their specific past usages or intended applications, are those illustrated by FIGS. 34-43 respectively. As exemplified by FIG. 34, a tubular conduit 550 is seen having a tubular wall 552 of fixed axial length; having two proximal open ends 554 and 556 which together generate the egress and exit to the interior of the conduit, a single internal lumen 558.

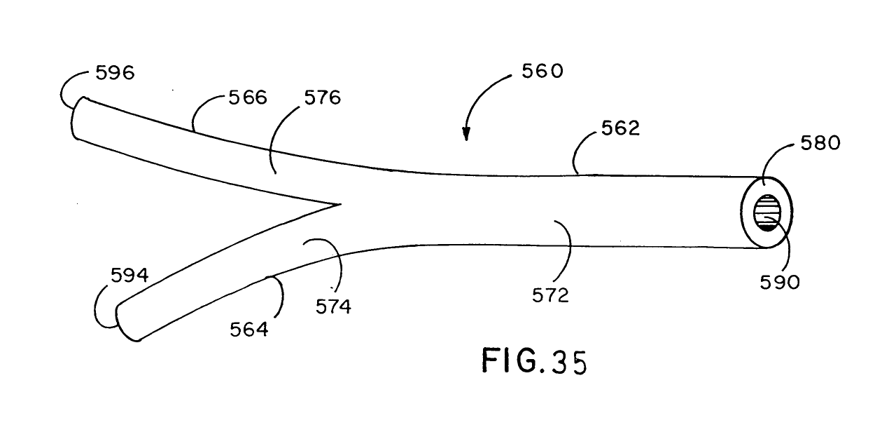

Another variation commonly known is illustrated by FIG. 35 which shows a conduit 560 having a central tubular wall portion 572 of fixed axial length; having two or more branches 574, 576 respectively which collectively form the proximal ends 596, 594 for entry into the internal volume of the conduit; and a single unbranched end 580. It will be appreciated and understood that FIGS. 34-43 are presented merely to show the overall general construction and relationship of parts present in each flexible tubular conduit suitable for use with the present methodology.

Also, in accordance with established principles of conventional construction, the axial length of the conduit may be composed of one or several layers in combination. In most multilayered constructions, one hollow tube is stretched over another to form a bond; and the components of the individual layers determine the overall characteristics for the conduit as a unitary construction. Some multilayered conduit structures comprise an inner tube of teflon, over which is another layer of nylon, woven Dacron, stainless steel or nitonol braiding. A tube of polyethylene or polyurethane is then heated and extruded over the two inner layers to form a bond as the third external layer. Other constructions may consist of a polyurethane inner core, covered by a layer of stainless steel, tungsten, polymer, carbon fiber or nitinol braiding, and a third external jacket layer formed of polyurethane.

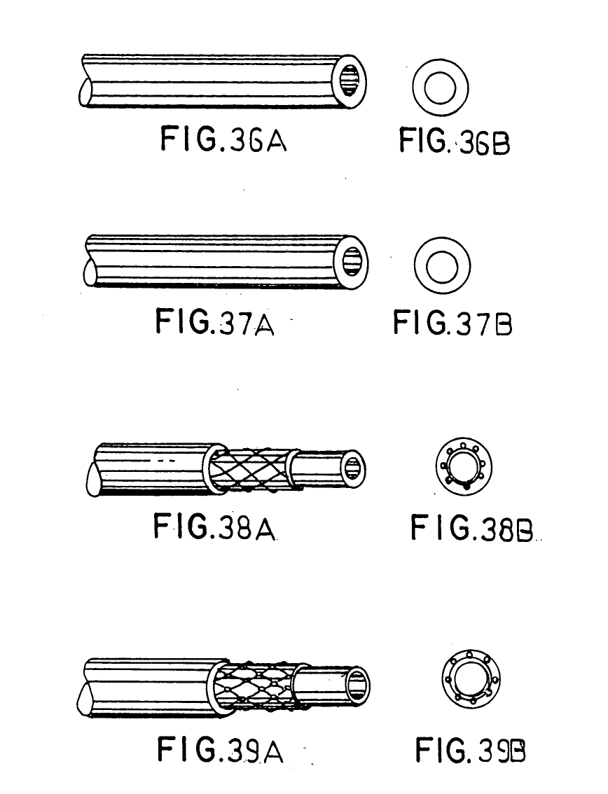

Several examples of basic conduit construction and design are illustrated by FIGS. 36-43 respectively. FIGS. 36A and 36B are perspective and cross-sectional views of a single tubular wall considered the standard minimum construction for a conduit. FIGS. 37A and 37B are perspective and cross-sectional views of a thin-walled design for a single layer extruded conduit. In comparison, FIGS. 38A and 38B are perspective and cross-sectional views of a standard multilayered construction having a braided stainless steel midlayer in its structure. Finally, FIGS. 39A and 39B are perspective and cross-sectional views of a thin-walled design for a multilayered conduit with a braided stainless steel middle layer.

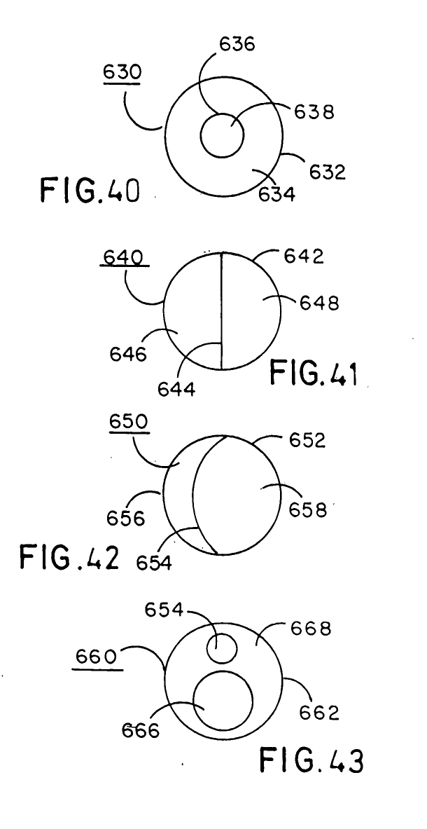

In addition, a number of different dual-lumen conduits are known today. These differ in size and spatial relationship between their individual lumens. The construction difference are illustrated by FIGS. 40-43 respectively which show different dual-lumen constructions for four tubular conduits having similar or identical overall diameter size.

As shown therein, FIG. 40 shows a dual-lumen conduit 630 wherein a first external tubular wall 632 provides an outer lumen volume 634 into which a second internal tubular wall 636 has been co-axially positioned to provide an inner lumen volume 638. Clearly, the construction of conduit 630 is a co-axial design of multiple tubular walls spaced apart and co-axially spaced but separate internal lumens of differing individual volumes.

In comparison, FIG. 41 shows a second kind of construction and design by dual-lumen conduit 640 having a single external tubular wall 642; and an centrally disposed inner septum 644 which divides the interior tubular space into two approximately equally lumen volumes 646 and 648 respectively. Thus, in this construction, the diameter, length, and volume of internal lumen 646 is effectively identical to the diameter, length and volume of internal lumen 640; and both of these exist and are contained within a single, commonly-shared, tubular wall.

A third kind of construction is illustrated by FIG. 42 and shows an alternative kind of construction and design. As seen in FIG. 42, the dual-lumen conduit 656 has a single external tubular wall 652; and contains an asymmetrically positioned internal divider 650 which divides the interior tubular space into two unequal and different lumen volumes 650 and 658 respectively. Thus, in this alternative construction, the discrete volume of internal lumen 650 is markedly smaller than the volume of the adjacently positioned internal lumen 658; and yet both of these internal lumens 650 and 658 exist in, are adjacently positioned, and are both contained within a commonly-shared single tubular wall.

A fourth construction and design for a dual-lumen conduit is presented by FIG. 43 which shows a conduit 660 having a single external tubular wall 662 of relatively large size and thickness. Within the material substance 668 of the tubular wall 660 are two discrete bore holes 664 and 666 of differing diameters which serve as two internal lumens of unequal volume. Internal lumen 664 is clearly the smaller while internal lumen 666 is far greater in spatial volume. Yet each internal lumen volume 664 and 666 is adjacent to the other, lies in parallel, and follows the other over the axial length of the conduit.

In general, the tubular body conduit is flexible over most of its length and may have one or more bends or curves towards the ends. Conventional practice also permits using a number of differently formed ends or tips which vary in design and appearance. Accordingly, for purposes of practicing the present invention, any construction of the tubular conduit whether having one or more curves, or none; and whether or not there is more than one designed portal for exiting or entering the lumen or multiple lumens are all considered conventional variations in construction design. Any and all of these designs and constructions are therefore deemed to be encompassed completely and to lie within the general scope of construction suitable for use with the present invention.

The present invention is not to be restricted in form nor limited in scope except by the claims appended hereto.

What I claim is:

1. A method for surgically attaching a ventricular assist device to the circulatory system of a living subject, said method comprising the steps of:
obtaining a ventricular assist device comprised of a housing for directing blood flow, a pump able to receive and convey blood, a first conduit of fixed dimensions and configuration having two open ends and at least one internal lumen for directing the flow of blood to said pump, a second conduit of fixed dimensions and configuration having two open ends and at least one internal lumen for conveying blood from said pump, and a source of power for the operation of said pump;
joining a linking connector to an end of said first conduit to form a prepared conduit for accessing and directing blood flow, said linking connector being of determined dimensions and configuration, being deformable on-demand, and being suitable for passage through an aperture;
acquiring a piercing introducer assembly suitable for the introduction of a prepared conduit to the vascular system of a living subject, said introducer assembly comprising:
a perforator instrument comprised of
(i) at least one elongated supporting shaft of predetermined overall dimensions and axial configuration,
(ii) a handle attached at one end to said supporting shaft; and
(iii) a perforating headpiece integrally joined to the other end of said supporting shaft, s id perforating headpiece comprising a perforating tip, a penetrating body, and a base aspect, and
(iv) conduit controlling means disposed adjacent to said perforating headpiece on said supporting shaft of said perforator instrument;
attaching said linking connector of said prepared conduit to a chamber of the heart in the living subject using said piercing introducer assembly such that said linking connector of said prepared inflow conduit passes through an aperture in the heart and deforms within a chamber of the heart, thereby securing said conduit to the interior of heart chamber and placing said secured conduit in blood flow communication with the interior of the heart chamber;
surgically affixing said second conduit to the vascular system of the living subject through a surgical means; and
connecting an end of said secured conduit and an end of said surgically affixed conduit to said pump of said ventricular assist device.

2. The method as recited by claim 1 wherein said first conduit is an inflow conduit and said second conduit is an outflow conduit.

3. The method as recited by claim 1 wherein second conduit comprises a linking connector joined to one end of said conduit.

4. The method as recited in claim 1 wherein said heart chamber is the left ventricle of the heart and said blood vessel is the aorta.

5. The method as recited in claim 1 wherein said heart chamber is the right ventricle of the heart and said blood vessel is the pulmonary artery.

6. A method for surgically attaching a ventricular assist device to the circulatory system of a living subject, said method comprising the steps of:
obtaining a ventricular assist device comprised of a housing for directing blood flow, a pump able to receive and convey blood, an inflow conduit of fixed dimensions and configuration having two open ends and at least one internal lumen for directing the flow of blood to said pump, an outflow conduit of fixed dimensions and configuration having two open ends and at least one internal lumen for conveying blood from said pump, and a source of power for the operation of said pump;
joining a linking connector to an end of said inflow conduit to form a prepared inflow conduit for accessing and directing blood flow, said linking connector being of determined dimensions and configuration, being deformable on-demand, and being suitable for passage through an aperture in and deformation within a first blood vessel, whereby said deformation serves to secure said prepared inflow conduit to a first blood vessel and places said secured inflow conduit in fluid flow communication with the interior of a first blood vessel;
joining a linking connector to an end of said outflow conduit to form a prepared outflow conduit for redirecting and conveying blood flow, said linking connector being of determined dimensions and configuration, being deformable on-demand, and being suitable for passage through an aperture in and deformation within a second blood vessel, whereby said deformation serves to secure said prepared outflow conduit to a second blood vessel and places said secured outflow conduit in fluid flow communication with the interior of a second blood vessel;
acquiring a catheterless, piercing introducer assembly suitable for the introduction and sutureless juncture of a prepared conduit to the vascular system of a living subject, said introducer assembly comprising:
a perforator instrument comprised of
(i) at least one elongated supporting shaft of predetermined overall dimensions and axial configuration,
(ii) a handle attached at one end to said supporting shaft; and
(iii) a perforating headpiece integrally joined to the other end of said supporting shaft, said perforating headpiece comprising a perforating tip, a penetrating body, and a base aspect, and
(iv) conduit controlling means disposed adjacent to said perforating headpiece on said supporting shaft of said perforator instrument;
attaching said linking connector of said prepared inflow conduit to a first blood vessel in the living subject using said piercing introducer assembly such that said linking connector of said prepared inflow conduit passes through an aperture in and deforms within a first blood vessel, thereby securing said inflow conduit to the interior of the first blood vessel and placing said secured inflow conduit in blood flow communication with the interior of a first blood vessel;
affixing said linking connector of said prepared outflow conduit to a second blood vessel in the living subject using said piercing introducer assembly such that said linking connector of said prepared outflow conduit passes through an aperture in and deforms within said second blood vessel, thereby securing said outflow conduit to the interior of the second blood vessel and placing said secured outflow conduit in blood flow communication with the interior of the second blood vessel; and
connecting an-end of said secured inflow conduit and an end of said secured outflow conduit to said pump of said ventricular assist device.

7. The method as recited in claim 1 or 6 wherein said ventricular assist device is selected from the group consisting of the HeartMate ventricular assist device, the Thoratec assist device, the Novacor ventricular assist device, the MicroMed (DeBakey) device, the Arrow LionHeart device, and the Levitronics device.

8. The method as recited in claim 1 or 6 wherein said perforator instrument of said piercing introducer assembly further comprises
a volumetric shaft having two open ends and at least one sidewall of determinable dimensions, said sheath being
(1) sized at one open end for on-demand placement adjacent to and aligned closure with said perforating headpiece of said perforator instrument,
(2) substantially annular in configuration over its axial length, and
(3) adapted for protective positioning around and volumetric spatial envelopment of at least a portion of said supporting shaft extending from said perforating headpiece of said perforator instrument, said sheath providing a protective covering for said enveloped spatial volume then surrounding said supporting shaft; and
position holding means attachable to and detachable from said volumetric sheath and said supporting shaft of said perforator instrument for holding said volumetric sheath and the enveloped spatial volume at a set position around said supporting shaft of said perforator instrument.

9. The method as recited in claim 1 or 6 wherein said supporting shaft of said introducer assembly is hollow over at least a portion of its length.

10. The method as recited in claim 1 or 6 wherein said conduit controlling means of said introducer assembly comprises an inflatable and deflatable on-demand balloon appliance disposed adjacent to said perforating headpiece.

11. The method as recited in claim 1 or 6 wherein said linking connector joined to said conduit is formed of a shape-memory alloy.

12. The method as recited in claim 1 or 6 wherein said linking connector joined to said conduit is formed of a super-elastic alloy.

13. The method as recited in claim 1 or 6 wherein said linking connector is a wire meshwork.

14. The method as recited in claim 1 or 6 wherein said conduit comprises a tube formed of a synthetic material.

15. The method as recited in claim 1 or 6 wherein said conduit further comprises a tube formed of naturally occurring matter.

16. The method as recited in claim 1 or 6 wherein said conduit further comprises one or more valves.

17. The method as recited in claim 1 or 6 which incorporates the use of a stabilizing ring to hold the conduit to the heart or vessel.

18. The method as recited in claim 1 or 6 where the connection is sutureless.
